SEQUENCE LISTING (28 sequences)

SEQ ID NO: 1 - protein, 60 aa, Artificial Sequence
  Synthetic Peptide
ARTKQTARKSTGGKAPRKQLATKAARKSAPATGESKKPHRYRPGTAALREIRRYQKSTEL

SEQ ID NO: 2 - protein, 60 aa, Artificial Sequence
  Synthetic Peptide
SGRGKGGKGLGKGGAKRHRKVLRDNIQGITKPAIRRLARRGGVKRISGLIYEETRGVLKV

SEQ ID NO: 3 - protein, 60 aa, Artificial Sequence
  Synthetic Peptide
SETAPAAPAAPAPAEKTPVKKKARKSAGAAKRKASGPPVSELITKAVAASKERSGVSLAA

SEQ ID NO: 4 - protein, 60 aa, Artificial Sequence
  Synthetic Peptide
SETAPAAPAAPAPAEKTPVKKKARKAAGGAKRKTSGPPVSELITKAVAASKERSGVSLAA

SEQ ID NO: 5 - protein, 18 aa, Artificial Sequence
  Synthetic Peptide
VKKKARKSAGAAKRKASG

SEQ ID NO: 6 - protein, 4 aa, Artificial Sequence
  Synthetic Peptide
ARTK

SEQ ID NO: 7 - protein, 5 aa, Artificial Sequence
  Synthetic Peptide
ARTKQ

SEQ ID NO: 8 - protein, 6 aa, Artificial Sequence
  Synthetic Peptide
ARTKQT

SEQ ID NO: 9 - protein, 7 aa, Artificial Sequence
  Synthetic Peptide
ARTKQTA

SEQ ID NO: 10 - protein, 8 aa, Artificial Sequence
  Synthetic Peptide
ARTKQTAR

SEQ ID NO: 11 - protein, 9 aa, Artificial Sequence
  Synthetic Peptide
ARTKQTARK

SEQ ID NO: 12 - protein, 4 aa, Artificial Sequence
  Synthetic Peptide
RTKQ

SEQ ID NO: 13 - protein, 5 aa, Artificial Sequence
  Synthetic Peptide
RTKQT

SEQ ID NO: 14 - protein, 4 aa, Artificial Sequence
  Synthetic Peptide
TKQT

SEQ ID NO: 15 - protein, 4 aa, Artificial Sequence
  Synthetic Peptide
KQTA

SEQ ID NO: 16 - protein, 22 aa, Artificial Sequence
  Synthetic Peptide
RKQLATKAARKSAPATGGVKKP

SEQ ID NO: 17 - protein, 21 aa, Artificial Sequence
  Synthetic Peptide
ARTKQTARKSTGGKAPRKQLA

SEQ ID NO: 18 - protein, 21 aa, Artificial Sequence
  Synthetic Peptide
LGKGGAKRHRKVLRDNIQGIT

SEQ ID NO: 19 - protein, 18 aa, Artificial Sequence
  Synthetic Peptide
VKKKARKSAGAAKRKASG

SEQ ID NO: 20 - protein, 21 aa, Artificial Sequence
  Synthetic Peptide
ARTKQTARKSTGGKAPRKQLA

SEQ ID NO: 21 - protein, 21 aa, Artificial Sequence
  Synthetic Peptide
ARTKQTARKSTGGKAPRKQLA

SEQ ID NO: 22 - protein, 21 aa, Artificial Sequence
  Synthetic Peptide
ARTKQTARKSTGGKAPRKQLA

SEQ ID NO: 23 - protein, 21 aa, Artificial Sequence
  Synthetic Peptide
ARTKQTARKSTGGKAPRKQLA

SEQ ID NO: 24 - protein, 21 aa, Artificial Sequence
  Synthetic Peptide
ARTKQTARKSTGGKAPRKQLA

SEQ ID NO: 25 - protein, 21 aa, Artificial Sequence
  Synthetic Peptide
ARTKQTARKSTGGKAPRKQLA

SEQ ID NO: 26 - protein, 22 aa, Artificial Sequence
  Synthetic Peptide
RKQLATKAARKSAPATGGVKKP

SEQ ID NO: 27 - protein, 21 aa, Artificial Sequence
  Synthetic Peptide
ARTKQTARKSTGGKAPRKQLA

SEQ ID NO: 28 - protein, 21 aa, Artificial Sequence
  Synthetic Peptide
LGKGGAKRHRKVLRDNIQGIT

(19) United States
(12) Patent Application Publication
(10) Pub. No.: US 2011/0021362 A1
(43) Pub. Date: Jan. 27, 2011

(54) AGENTS FOR STIMULATING ACTIVITY OF METHYL MODIFYING ENZYMES AND METHODS OF USE THEREOF

(75) Inventors: Patrick Trojer, Burlington, MA (US); Fei Lan, Brookline, MA (US)

Correspondence Address: CHOATE, HALL & STEWART LLP, TWO INTERNATIONAL PLACE, BOSTON, MA, 02110, US

(73) Assignee: CONSTELLATION PHARMACEUTICALS, Cambridge, MA (US)

(21) Appl. No.: 12/839,594

(22) Filed: Jul. 20, 2010

Related U.S. Application Data
(60) Provisional application No. 61/227,031, filed on Jul. 20, 2009.

(51) Int. Cl. C40B 30/00; C12Q 1/68; G01N 33/566

(52) U.S. Cl. 506/7; 435/6; 435/7.1

(57) ABSTRACT

Agents for stimulating activity of methyl modifying enzymes and methods of using the enzymes in assays to identify enzyme modulators are provided herein.

### RELATED APPLICATIONS

This application is related to and claims priority under 35 U.S.C. §119(e) to U.S. provisional patent application No. 61/227,031, filed Jul. 20, 2009 (“the '031 application”). The entire contents of the '031 application are incorporated herein by reference.

### BACKGROUND OF THE INVENTION

Eukaryotic chromatin is composed of macromolecular complexes called nucleosomes. A nucleosome has 147 base pairs of DNA wrapped around a protein octamer having two subunits of each of histone protein H2A, H2B, H3, and H4. Histone proteins are subject to post-translational modifications which in turn affect chromatin structure and gene expression. One type of post-translational modification found on histones is methylation of lysine and arginine residues. Histone methylation plays a critical role in the regulation of gene expression in eukaryotes. Methylation affects chromatin structure and has been linked to both activation and repression of transcription (Zhang and Reinberg, Genes Dev. 15:2343-2360, 2001). Enzymes that catalyze attachment and removal of methyl groups from histones are implicated in gene silencing, embryonic development, cell proliferation, and other processes.

### SUMMARY OF THE INVENTION

The present disclosure encompasses the recognition that methyl modifying enzymes are an attractive target for modulation, given their role in the regulation of diverse biological processes. The present disclosure provides methods and compositions to facilitate identification of modulators of these enzymes by enhancing their activity in vitro. For example, according to the present disclosure, it has been discovered that methylase and demethylase activity can be stimulated by addition of peptides to enzymatic reactions or by introducing particular modifications on substrate molecules, thereby stimulating enzymatic activity and/or changing target site specificity, and in this context providing a more robust platform for evaluating candidate agents for inhibition and/or activation of enzymatic activity. In particular embodiments, the present disclosure provides agents that stimulate activity of histone methyl modifying enzymes, including histone methylases and histone demethylases. Stimulating agents for histone methylases and demethylases include methylated histone peptides (e.g., synthetic peptides composed of amino acids mimicking the sequence of distinct regions of histone proteins).

Accordingly, in one aspect, the present disclosure features a method of evaluating a test compound including, for example: contacting a methyl modifying enzyme and a substrate with a test compound in the presence of a stimulating agent; evaluating activity of the methyl modifying enzyme on the substrate in the presence of the test compound, relative to a control, wherein a change in activity of the methyl modifying enzyme in the presence of the test compound, e.g., relative to a control, indicates that the test compound is a modulator of the methyl modifying enzyme. In some embodiments, the invention provides high-throughput formats for performing such methods, for example allowing simultaneous assessment of at least 5, 10, 15, 20, 25, 30, 35, 40, 45, 50, 55, 60, 65, 70, 75, 80, 85, 90, 100 or more (and in some embodiments several 100s or 1000s) of reactions.

In some embodiments, a methyl modifying enzyme comprises a histone methyl modifying enzyme. In some embodiments, a methyl modifying enzyme comprises a methylase (e.g., a human histone methylase, e.g., a human histone methylase in Table 1A). In some embodiments, a methyl modifying enzyme comprises a demethylase (e.g., a human histone demethylase, e.g., a human histone demethylase in Table 1B).

A substrate can include a peptide (e.g., a histone peptide), a polypeptide (e.g., histone polypeptide), a histone dimer (e.g., an H2A-H2B dimer), a histone tetramer (e.g., an H3-H4 tetramer), a histone octamer, a nucleosome, an oligonucleosome, chromatin (e.g., in the presence or absence of histone H1 isotypes), or a combination thereof.

A stimulating agent can include a peptide, e.g., a methylated peptide. In some embodiments, a stimulating agent comprises a peptide 4-60 amino acids in length. In some embodiments, a methylated peptide comprises one or more methylated lysine residues. In some embodiments, a methylated peptide comprises one or more tri-methylated lysine residues. In some embodiments, a methylated peptide comprises one or more di-methylated lysine residues. In some embodiments, a methylated peptide comprises one or more mono-methylated lysine residues.

In some embodiments, a stimulating agent comprises a histone peptide, e.g., a methylated histone peptide. In some embodiments, a methylated histone peptide comprises a methylated histone H3 peptide, a methylated histone H4 peptide, or a methylated histone H1 peptide.

In some embodiments, a methylated histone peptide comprises one or more tri-methylated lysine residues, one or more di-methylated lysine residues, and/or one or more mono-methylated lysine residues.

In some embodiments, a methylated histone peptide comprises at least four consecutive amino acids of the following H3 histone peptide sequence: ARTKQTARKSTGGKAPRKQLATKAARKSAPATGESKKPHRYRPGTAALREIRRYQKST EL (SEQ ID NO:1). In some embodiments, an H3 histone peptide is methylated on one or more of the following lysine residues: K4, K9, K27, and K36. In some embodiments, a H3 histone peptide is methylated on K27. In some embodiments, an H3 histone peptide is methylated on K9.

In some embodiments, a methylated histone peptide comprises at least four consecutive amino acids of the following H4 histone peptide sequence: SGRGKGGKGLGKGGAKRHRKVLRDNIQGITKPAIRRLARRGGVKRISGLIYEETRGVLK V (SEQ ID NO:2). In some embodiments, an H4 histone peptide is methylated on K20.

In some embodiments, a methylated histone peptide comprises at least four consecutive amino acids of the following H1 histone peptide sequence: SETAPAAPAAPAPAEKTPVKKKARKSAGAAKRKASGPPVSELITKAVAASKERSGVSLA A (SEQ ID NO:3).

In some embodiments, an H1 histone peptide is methylated on K26.

In some embodiments, a stimulating agent is present in an amount which stimulates activity of the methyl modifying enzyme at least 2-fold, at least 5-fold, or at least 10-fold.

A test compound can include a small molecule, a peptide, and/or a nucleic acid.

In some embodiments of a method provided herein, a methyl modifying enzyme and substrate are contacted with a library of test compounds, and a change in activity of the methyl modifying enzyme in the presence of the library, relative to a control, indicates that the library comprises a modulator of the methyl modifying enzyme. A method can further include selecting the modulator from the library.

In another aspect, the present disclosure features reaction mixture including, for example: a substrate of a methyl modifying enzyme; and a stimulating agent, wherein the stimulating agent is present in an amount sufficient to increase activity of a methyl modifying enzyme. The reaction mixture can further include a methyl modifying enzyme.

A methyl modifying enzyme can include a histone methyl modifying enzyme. A methyl modifying enzyme can include a methylase or a demethylase.

In some embodiments, a substrate comprises a peptide (e.g., a histone peptide), a polypeptide (e.g., a histone polypeptide), a nucleosome, an oligonucleosome, chromatin, or a combination thereof.

A stimulating agent can include a peptide, e.g., a methylated peptide. In some embodiments, a stimulating agent comprises a peptide 4-60 amino acids in length. In some embodiments, a methylated peptide comprises one or more methylated lysine residues. In some embodiments, a methylated peptide comprises one or more tri-methylated lysine residues. In some embodiments, a methylated peptide comprises one or more di-methylated lysine residues. In some embodiments, a methylated peptide comprises one or more mono-methylated lysine residues.

In some embodiments, a stimulating agent comprises a histone peptide, e.g., a methylated histone peptide. In some embodiments, a methylated histone peptide comprises a methylated histone H3 peptide, a methylated histone H4 peptide, a methylated histone H1 peptide.

In some embodiments, a methylated histone peptide comprises one or more tri-methylated lysine residues, one or more di-methylated lysine residues, and/or one or more mono-methylated lysine residues.

In some embodiments, a methylated histone peptide comprises at least four consecutive amino acids of the following H3 histone peptide sequence: ARTKQTARKSTGGKAPRKQLATKAARKSAPATGESKKPHRYRPGTAALREIRRYQKST EL (SEQ ID NO:1). In some embodiments, an H3 histone peptide is methylated on one or more of the following lysine residues: K4, K9, K27, and K36. In some embodiments, a H3 histone peptide is methylated on K27. In some embodiments, an H3 histone peptide is methylated on K9.

In some embodiments, a methylated histone peptide comprises at least four consecutive amino acids of the following H4 histone peptide sequence: SGRGKGGKGLGKGGAKRHRKVLRDNIQGITKPAIRRLARRGGVKRISGLIYEETRGVLK V (SEQ ID NO:2). In some embodiments, an H4 histone peptide is methylated on K20.

In some embodiments, a methylated histone peptide comprises at least four consecutive amino acids of the following H1 histone peptide sequence: SETAPAAPAAPAPAEKTPVKKKARKSAGAAKRKASGPPVSELITKAVAASKERSGVSLA A (SEQ ID NO:3).

In some embodiments, an H1 histone peptide is methylated on K26.

In some embodiments, a stimulating agent is present in an amount which stimulates activity of the methyl modifying enzyme at least 2-fold, at least 5-fold, or at least 10-fold.

In another aspect, the present disclosure provides a composition comprising a stimulating agent described herein.

According to the present disclosure, stimulating agents confer various benefits. For example, the presence of a stimulating agent can increase sensitivity of an assay. Alternatively or additionally, the presence of a stimulating agent can allow one to use less enzyme in assays (e.g., five, 10, 25, 50, 100 fold less than needed in the absence of a stimulating agent), thereby reducing costs and/or facilitating adaptation to high throughput formats. In some embodiments, a stimulating agent mimics an interaction encountered by an enzyme in vivo. In such embodiments, modulation of enzyme activity in the presence of a stimulating agent can reflect modulation in a more physiologically relevant state. Compounds identified under such conditions may be found to have greater specificity and/or superior activity in vivo.

Details of one or more embodiments of the invention are set forth in the accompanying drawings and the description below. Other features, objects, and advantages of the invention will be apparent from the description and drawings, and from the claims. All cited patents, patent applications, and references (including references to public sequence database entries) are incorporated by reference in their entireties for all purposes.

### BRIEF DESCRIPTION OF THE DRAWING

FIG. 1A is a schematic depiction of a recombinant Polycomb Repressive Complex 2 (rPRC2) complex, including EZH2, EED, SUZ12, RBAP46, and RBAP48 subunits.

FIG. 1B shows silver staining and Western blot analysis of rPRC2 preparation used in examples described herein.

FIG. 1C shows analysis of H3, H2A/H2B, H4, and [³H]-H3 labeled substrate from reactions with rPRC2 and wild type histone H3 (wt) or H3 having a K27A substitution (H3K27A). Fluorographic analysis is shown in the top panel. Coomassie staining is shown in the bottom panel.

FIG. 2A shows fluorographic analysis of [³H]-H3 in wild type histone H3 (H3 wt), H3K27A, biotin/avidin labeled H3 (Bio/Avi-H3), wild type octamers (octamers wt), octamers containing H3K27A, and Bio/Avi-octamers incubated with rPRC2. Coomassie staining is shown in the bottom panel.

FIG. 2B shows TopCount analysis of methylase reaction products shown in FIG. 2A.

FIG. 3A shows fluorographic analysis of [³H]-Bio/Avi-H3 in Bio/Avi-oligonucleosomes incubated with rPRC2. Coomassie staining is shown in the bottom panel.

FIG. 3B shows TopCount analysis of methylase reaction products shown in FIG. 3A.

FIG. 3C shows quantitative information for oligonucleosome substrates used in reactions shown in FIGS. 3A and 3B.

FIG. 3D is a graph of [³H]-cpm in methylase reactions shown in FIGS. 3A-3C using increasing concentrations of oligonucleosomes.

FIGS. 4A and 4B are graphs showing [³H]-cpm (FIG. 4A) and Michaelis-Menten data (FIG. 4B) for increasing concentrations of oligonucleosomes in methylase reactions.

FIG. 5 is a graph showing stimulation of rPRC2 methylase activity in the presence of unmodified H3 or the following: H3K4me3, H3K9me3, H3K27me3, H3K36me3, H3K79me3, H4K20me3, and H1.4K26me3 peptides.

FIG. 6A shows fluorographic analysis of [³H]-EZH2 and [³H]-rAvi-H3 in a methylase assay using rPRC2 in the presence of H3K27me3, H3K27me0, H3K9me3, H4K20me3, or no stimulating agent. Bio/Avi-H3 was used as substrate. Coomassie staining is shown in the bottom panel.

FIG. 6B is a graph of TopCount analysis of reactions shown in FIG. 6C.

FIG. 6C shows fluorographic analysis of [³H]-EZH2 and [³H]-rAvi-H3 in methylase assays using rPRC2 in the presence of H3K27me3, H3K27me0, H3K9me3, H4K20me3, or no stimulating agent. Bio/Avi-oligonucleosomes were used as substrate. Coomassie staining is shown in the bottom panel.

FIG. 6D is a graph of photostimulated luminescence (PSL) for reactions shown in FIG. 6C.

FIG. 7A shows fluorographic analysis of [³H]-Bio/Avi-H3 in methylase assays using rPRC2 in the presence of H3K27me3, H3K27me2, H3K27me1, H3K27me0, H3K9me3, or H4K20me3 peptides. Coomassie staining is shown in the bottom panel.

FIG. 7B is a graph of TopCount analysis of reactions shown in FIG. 7A.

FIG. 8A is a graph showing a time course of methylation in an assay using rPRC2 in the presence of an excess amount of a stimulating agent, H3K27me3.

FIG. 8B is a graph showing a time course of methylation in an assay using rPRC2 in the presence of a limiting amount of a stimulating agent, H3K27me3 (1.24 μM).

FIG. 8C shows conditions used for time course assays shown in FIGS. 8A and 8B.

FIG. 9 is a graph showing a time course of methylation in an assay using rPRC2.

FIG. 10A shows conditions used for methylase assays depicted in FIGS. 10A and 10B.

FIGS. 10B and 10C are graphs showing titration of rPRC2 enzyme using oligonucleosomes as a substrate. FIG. 10B shows results from Day 1, using robotics. FIG. 10C shows results from Day 2, using robotics.

FIG. 11A is an analysis of Nuclear SET domain-containing 2 (NSD2) protein from 293 cells.

FIG. 11B shows fluorographic analysis of [³H]-H3 in methylase assays using NSD2 enzyme and octamers or nucleosomes as a substrate. Coomassie staining is shown in the bottom panel.

FIG. 12A is a graph showing counts per minute of labeled SAM from methylase assays using NSD2 in the presence of various histone peptides.

FIG. 12B is a graph showing fold increase in NSD2 activity in the presence of different concentrations H3K36me2 or H3K79me2.

### DEFINITIONS

Characteristic sequence element: As used herein, the term “characteristic sequence element” or “sequence element” refers to a stretch of contiguous amino acids, typically 5 amino acids, e.g., at least 5-50, 5-25, 5-15, or 5-10 amino acids, that shows at least about 80%, 85%, 90%, 95%, 96%, 97%, 98%, 99%, or 100% identity with another polypeptide. In some embodiments, a characteristic sequence element participates in or confers function on a polypeptide.

Corresponding to: As used herein, the term “corresponding to” is often used to designate the position/identity of an amino acid residue in a peptide or polypeptide (e.g., in a histone peptide). Those of ordinary skill will appreciate that, for purposes of simplicity, a canonical numbering system (based on wild type histone polypeptides) is utilized herein, so that an amino acid “corresponding to” a lysine residue at position 4 (K4) of histone H3 (also referred to as “H3K4”), for example, need not actually be the 4th amino acid in a particular histone peptide amino acid chain but rather corresponds to the residue found at position 4 in a wild type polypeptide (e.g., in a wild type histone polypeptide); those of ordinary skill in the art readily appreciate how to identify corresponding amino acids.

Demethylase: A “demethylase”, as used herein, refers to an enzyme that removes a methyl group or multiple methyl groups from a substrate. The term refers to catalytic demethylase subunits as well as protein complexes containing the catalytic subunits. In some embodiments, a demethylase is a protein demethylase, i.e., an enzyme that removes methyl groups from a polypeptide substrate. In some embodiments, a demethylase is a histone demethylase, i.e., an enzyme that removes methyl groups from a histone polypeptide substrate.

Histone peptide: The term “histone peptide” as used herein, refers to a peptide that has structural and/or functional similarity to a portion of a wild type histone polypeptide (and includes portions of histone polypeptides) (i.e., a histone peptide has a sequence that is not a full-length histone polypeptide sequence). In some embodiments, a histone peptide has an amino acid sequence that is substantially identical to that of a portion of a wild type histone polypeptide. In some embodiments, a histone peptide has an amino acid sequence that is substantially identical to that of an N-terminal portion of a histone polypeptide. In some embodiments, a histone peptide is less than 60, 50, 40, 30, 20, 10, or fewer amino acids long. In some embodiments, a histone peptide is more than 5, 6, 7, 8, 9, 10, 11, 12, 13, 14, 15, 16, 17, 18, 19, 20, 21, 22, 23, 24, 25, 26, 27, 28, 29, 30, or more amino acids long. In some embodiments, a histone peptide is between about 20 and about 60 amino acids long. In some embodiments, a histone peptide is between about 10 and about 50 amino acids long. In some embodiments, a histone peptide has an amino acid sequence that includes one or more lysine residues. In some embodiments, a histone peptide has an amino acid sequence that includes one or more methylated (e.g., mono-, di-, and/or tri-methylated) lysine residues. In some embodiments, a histone peptide has an amino acid sequence that includes a plurality of sequence elements, each of which is found in a natural histone polypeptide. In some embodiments, a histone peptide has an amino acid sequence that includes a plurality of sequence elements that are found in (or share substantially identity with sequence elements that are found in) a plurality of different natural histone polypeptides.

Methyl modifying enzyme: The term “methyl modifying enzyme”, as used herein, refers to an enzyme that catalyzes transfer of a methyl group from one molecule to another. Methyl modifying enzymes include methylases (e.g., methylases that attach methyl groups to polypeptide substrates) and demethylases (e.g., demethylases that remove methyl groups from polypeptide substrates). Methyl modifying enzymes include enzymes having a full length sequence, enzymes having a portion of a full length sequence, and/or partial enzyme complexes that retain enzymatic activity.

Methylase: A “methylase”, as used herein, refers to an enzyme that attaches a methyl group to a substrate. The term refers to catalytic methylase subunits as well as protein complexes containing the catalytic subunits. Methylases are also referred to as methyltransferases. In some embodiments, a methylase is a protein methylase, i.e., an enzyme that attaches methyl groups to polypeptide substrate. In some embodiments, a methylase is a histone methylase, i.e., an enzyme that attaches methyl groups to a histone polypeptide substrate.

Methylated: The term “methylated”, as used herein, refers to the presence of one or more methyl groups on a molecule (e.g., peptide). In some embodiments, a methylated peptide has one methylated amino acid. In some embodiments, a methylated peptide has more than one methylated amino acid. In some embodiments, an amino acid residue on a methylated peptide has one or more methyl groups (i.e., a residue is di- or tri-methylated).

Polypeptide: The term “polypeptide”, as used herein, generally has its art-recognized meaning of a polymer of at least three amino acids. However, the term is also used to refer to specific functional classes of polypeptides, such as, for example, methylase polypeptides, demethylase polypeptides, histone polypeptides, etc. For each such class, the present specification provides several examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides described herein.

Stimulating agent: The term “stimulating agent”, as used herein, refers to an agent that increases activity of a methyl modifying enzyme. A stimulating agent of a methylase enzyme increases methylase activity of the enzyme. A stimulating agent of a demethylase enzyme increases demethylase activity of the enzyme. In some embodiments, a stimulating agent is a peptide 4-60 amino acids in length. In some embodiments, a stimulating agent is a methylated peptide 4-60 amino acids in length. A stimulating agent can include, or consist of, a peptide sequence (e.g., a methylated peptide sequence) of a histone polypeptide, such as an H3, H1, or H4 polypeptide. Stimulating agents can include peptides (e.g., methylated peptides) having natural and/or non-natural amino acids. Stimulating agents can include modifications such one or more labels. In some embodiments, a stimulating agent is biotinylated. In some embodiments, enzyme activity is stimulated two, three, four, five, ten, twenty, fifty-fold, or more, in the presence of a stimulating agent.

Substantial identity: The term “substantial identity” of amino acid sequences (and of polypeptides having these amino acid sequences) typically means sequence identity of at least 40% compared to a reference sequence as determined by comparative techniques known in the art. For example, a variety of computer software programs are well known for particular sequence comparisons. In some embodiments, the BLAST is utilized, using standard parameters, as described. In some embodiments, the preferred percent identity of amino acids can be any integer from 40% to 100%. In some embodiments, sequences are substantially identical if they show at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical residues in corresponding positions. In some embodiments, polypeptides are considered to be “substantially identical” when they share amino acid sequences as noted above except that residue positions which are not identical differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

As mentioned above, one example of an algorithm that is suitable for determining percent sequence identity and sequence similarity is the BLAST algorithm, which is described in Altschul et al., 1977, Nuc. Acids Res. 25:3389-3402. BLAST is used, with the parameters described herein, to determine percent sequence identity for the nucleic acids and proteins of the present disclosure. Software for performing BLAST analysis is publicly available through the National Center for Biotechnology Information (available at the following internet address: ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Substrate: A “substrate” as used herein to describe substrates of a methyl modifying enzyme, refers to any peptide, polypeptide, or molecular complex that can be modified by activity of the enzyme. In general, a “substrate” of an enzyme, is an entity with which the enzyme specifically interacts (e.g., in the presence of other entities). Substrates of methyl modifying enzymes include peptides or polypeptides that have a site to which a methyl can be attached and/or removed. In some embodiments, a substrate of a methyl modifying enzyme comprises a histone peptide or histone polypeptide. In some embodiments, a substrate of a methyl modifying enzyme comprises a nucleosome. In some embodiments, a substrate of a methyl modifying enzyme comprises an oligonucleosome. In some embodiments, a substrate of a methyl modifying enzyme comprises chromatin.

Test compound: A “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole, e.g., between 5,000 to 500 grams per mole. The test compound can be naturally occurring (e.g., a herb or a nature product), synthetic, or both. Examples of macromolecules are proteins (e.g., antibodies, antibody fragments), protein complexes, and glycoproteins, nucleic acids, e.g., DNA, RNA (e.g., siRNA), and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heteroorganic or organometallic compounds.

Wild type: The term “wild-type”, when applied to a polypeptide (e.g., a histone polypeptide) has its art understood meaning and refers to a polypeptide whose primary amino acid sequence is identical to that of a polypeptide found in nature. As will be appreciated by those skilled in the art, a wild type polypeptide is one whose amino acid sequence is found in normal (i.e., non-mutant) polypeptides.

### DETAILED DESCRIPTION OF CERTAIN EMBODIMENTS

### Histone Methyl Modifying Enzymes

The present disclosure provides methods and compositions for identifying compounds that modulate activity of histone methyl modifying enzymes. Histone methyl modifying enzymes are key regulators of cellular and developmental processes. Such enzymes have modules that mediate binding to methylated residues. For example, multiple demethylases contain a Tudor domain (e.g., JMJD2C/GASC1) or a PHD domain (e.g., JARID1C/SMCX, PHF8). In some embodiments, stimulating agents described herein present one or more modifications recognized by a methyl binding domain of an enzyme of interest and provide a more physiological environment for the enzyme, thereby increasing its activity (e.g., by increasing substrate affinity).

One class of histone methylases is characterized by the presence of a SET domain, named after proteins that share the domain, Su(var)3-9, enhancer of zeste [E(Z)], and trithorax. A SET domain includes about 130 amino acids. SET domain-containing methylase families include SUV39H1, SET1, SET2, EZH2, RIZ1, SMYD3, SUV4-20H1, SET7/9, and PR-SET7/SET8 families (reviewed in Dillon et al., Genome Biol. 6:227, 2005). Members of a family typically include similar sequence motifs in the vicinity of and within the SET domain. The human genome encodes over 50 SET domain-containing histone protein methylases, any of which can be used in an assay described herein.

EZH2 is an example of a human SET-domain containing methylase. EZH2 associates with EED (Embryonic Ectoderm Development) and SUZ12 (suppressor of zeste 12 homolog) to form a complex known as PRC2 (Polycomb Group Repressive Complex 2) having the ability to tri-methylate histone H3 at lysine 27 (Cao and Zhang, Mol. Cell. 15:57-67, 2004). PRC2 complexes can also include RBAP46 and RBAP48 subunits. EZH2 overexpression is associated with aggressiveness of certain cancers such as breast cancer (Kleer et al., Proc. Nat. Acad. Sci. USA 100:11606-11611, 2003).

The lysine specificities of many histone methyltransferases have been characterized. For example SET7/9, SMYD3, and MLL1-5 are specific for H3K4. SUV39H1, DIM-5, and G9a are specific for H3K9. SET8 is specific for H4K20.

DOT1 is an example of a non-SET domain containing histone methylase. DOT1 methylates H3 on lysine 79.

Just as histone methylases have been shown to regulate transcriptional activity, chromatin structure, and gene silencing, demethylases have also been discovered which impact gene expression. LSD1 was the first histone lysine demethylase to be characterized. This enzyme displays homology to FAD-dependent amine oxidases and acts as a transcriptional corepressor of neuronal genes (Shi et al., Cell 119:941-953, 2004). Additional demethylases defining separate demethylase families have been discovered, including JHDM1 (or KDM2), JHDM2 (or KDM3), JMJD2 (or KDM4), JARID (or KDM5), JMJD3 (or KDM6), and JMJD6 families (Lan et al., Curr. Opin. Cell Biol. 20(3):316-325, 2008).

Demethylases act on specific lysine residues within substrate sequences and discriminate between the degree of methylation present on a given residue. For example, LSD1 removes mono- or dimethyl-groups from H3K4. Members of the JARID1A-D family remove trimethyl groups from H3K4. UTX and JMJD3 demethylate H3K27, counteracting effects of EZH2 methylase activity. Substrate specificities of other demethylases have been characterized (see Shi, Nat. Rev. 8:829-833, 2007).

Histone methyl modifying enzymes can be produced recombinantly or purified from a natural source. Histone methyl modifying enzymes are also commercially available. In some embodiments, a histone methyl modifying enzyme used in a method or composition described herein is a human enzyme. In some embodiments, a histone methyl modifying enzyme used in a method or composition described herein is a non-human enzyme (e.g., a murine, rat, bovine, equine, porcine, canine, chicken, zebrafish, chimpanzee, macaque, Drosophila, C. elegans, Xenopus, or Anopheles enzyme). Examples of human histone methylases and demethylases that can be used according to the present disclosure are listed in Tables 1A and 1B. Non-human homologs of enzymes shown in Tables 1A and 1B, as well as additional human and non-human methyl modifying enzymes are known and can also be used in/part of methods and compositions described herein.

TABLE 1A
Exemplary Methylases

| | | | GenBank |
|---|---|---|---|
| | | | Acc. No. |
| | | GenBank | (amino acid |
| Name | Alternative names | GeneID No. | seq.) |
| SET domain | Set1; KMT2F; Set1A; KIAA0339; SETD1A | 9739 | NP_055527.1 |
| containing 1A | | | |
| Myeloid/lymphoid or | HRX; TRX1; ALL-1; CXXC7; HTRX1; KMT2A; | 4297 | NP_005924.2 |
| mixed lineage | MLL1A; FLJ11783; MLL/GAS7; TET1-MLL; | | |
| leukemia associated | MLL; trithorax-like protein; | | |
| protein 1 | zinc finger protein HRX; | | |
| | MLL-AF4 der(11) fusion protein | | |
| myeloid/lymphoid or | ALR; MLL4; AAD10; TNRC21; CAGL114; | 8085 | NP_003473.3 |
| mixed-lineage | MLL2; | | |
| leukemia 2 | ALL1-related; | | |
| | trinucleotide repeat containing 21 | | |
| myeloid/lymphoid or | HALR; KMT2C; FLJ12625; FLJ38309; | 58508 | NP_733751.2 |
| mixed-lineage | KIAA1506; MGC119851; MGC119852; | | |
| leukemia 3 | MGC119853; DKFZp686C08112; MLL3; ALR- | | |
| | like protein; | | |
| | histone-lysine N-methyltransferase, H3 lysine-4 | | |
| | specific | | |
| myeloid/lymphoid or | MLL4; HRX2; MLL2; TRX2; WBP7; KIAA0304; | 9757 | NP_055542.1 |
| mixed-lineage | trithorax homologue 2; | | |
| leukemia 4 | WW domain binding protein 7; | | |
| | mixed lineage leukemia gene homolog 2 | | |
| myeloid/lymphoid or | MLL5; KMT2E; FLJ10078; FLJ14026; | 55904 | NP_061152.3 |
| mixed-lineage | HDCMC04P; MGC70452; MLL5 | | |
| leukemia 5 | | | |
| Absent, small or | ASH1; KMT2H; ASH1L1; FLJ10504; KIAA1420; | 55870 | NP_060959.2 |
| homeotic like 1 | ASH1L | | |
| Suppressor of | MG44; KMT1A; SUV39H; SUV39H1; | 6839 | NP_003164.1 |
| variegation 3-9 | H3-K9-HMTase 1; | | |
| homolog | OTTHUMP00000024298; | | |
| | Su(var)3-9 homolog 1; | | |
| | histone H3-K9; methyltransferase 1; | | |
| | histone-lysine N-methyltransferase, H3 lysine-9 | | |
| | specific 1 | | |
| suppressor of | KMT1B; FLJ23414; SUV39H2; | 79723 | NP_078946.1 |
| variegation 3-9 | OTTHUMP00000019186; | | |
| homolog 2 | OTTHUMP00000019187 | | |
| euchromatic histone- | GLP; KMT1D; DEL9q34; FP13812; FLJ12879; | 79813 | NP_001138999.1 |
| lysine N- | KIAA1876; EUHMTASE1; Eu-HMTase1; | | |
| methyltransferase 1 | bA188C12.1; DKFZp667M072; RP11-188C12.1; | | |
| | EHMT1GLP; KMT1D; DEL9q34; FP13812; | | |
| | FLJ12879; KIAA1876; EUHMTASE1; Eu- | | |
| | HMTase1; bA188C12.1; DKFZp667M072; RP11- | | |
| | 188C12.1; EHMT1; H3-K9-HMTase 5; | | |
| | G9a like protein; | | |
| | OTTHUMP00000022711; | | |
| | lysine N-methyltransferase 1D; | | |
| | histone H3-K9 methyltransferase 5; | | |
| | histone-lysine N-methyltransferase, H3 lysine-9 | | |
| | specific 5 | | |
| euchromatic histone- | G9A; BAT8; NG36; KMT1C; C6orf30; FLJ35547; | 10919 | NP_006700.3 |
| lysine N- | DKFZp686H08213; EHMT2; euchromatic histone- | | |
| methyltransferase 2 | lysine N-methyltransferase 2; | | |
| | protein G9a; | | |
| | H3-K9-HMTase 3; | | |
| | OTTHUMP00000029262; | | |
| | G9A histone methyltransferase; | | |
| | HLA-B associated transcript 8; | | |
| | lysine N-methyltransferase 1C; | | |
| | ankyrin repeat-containing protein; | | |
| | histone H3-K9 methyltransferase 3 | | |
| SET domain, | ESET; KG1T; KMT1E; KIAA0067; H3-K9- | 9869 | NP_001138887.1 |
| bifurcated 1 | HMTase4; SETDB1; lysine N-methyltransferase | | |
| | 1E; | | |
| | ERG-associated protein with a SET domain, | | |
| | ESET; | | |
| | histone-lysine N-methyltransferase, H3lysine-9 | | |
| | specific 4 | | |
| PR domain containing | RIZ; KMT8; RIZ1; RIZ2; MTB-ZF; | 7799 | NP_001007258.1 |
| 2, with ZNF domain | HUMHOXY1; PRDM2; retinoblastoma protein- | | |
| | binding zinc finger protein; | | |
| | OTTHUMP00000009642; | | |
| | OTTHUMP00000009687; | | |
| | MTE-binding protein; | | |
| | GATA-3 binding protein G3B; | | |
| | zinc-finger DNA-binding protein; | | |
| | retinoblastoma protein-interacting zinc finger | | |
| | protein | | |
| Enhancer of zeste | ENX1; KMT6; ENX-1; MGC9169; EZH2; lysine | 2146 | NP_004447.2 |
| homolog | N-methyltransferase 6 | | |
| SET domain | HYPB; SET2; HIF-1; KMT3A; HBP231; | 29072 | NP_054878.5 |
| containing 2 | HSPC069; p231HBP; FLJ16420; FLJ22472; | | |
| | FLJ23184; FLJ45883; FLJ46217; KIAA1732; | | |
| | SETD2; huntingtin yeast partner B; | | |
| | lysine N-methyltransferase 3A; | | |
| | SET domain-containing protein 2; | | |
| | huntingtin interacting protein 1; | | |
| | huntingtin-interacting protein B; | | |
| | histone-lysine N-methyltransferase SETD2 | | |
| nuclear receptor | STO; KMT3B; SOTOS; ARA267; FLJ10684; | 64324 | NP_071900.2 |
| binding SET domain | FLJ22263; FLJ44628; DKFZp666C163; NSD1; | | |
| protein 1 | androgen receptor-associated coregulator 267 | | |
| SET and MYND | KMT3C; HSKM-B; ZMYND14; MGC119305; | 56950 | NP_064582.2 |
| domain containing 2 | SMYD2; SET and MYND domain containing 2; | | |
| | OTTHUMP00000035134; | | |
| | zinc finger, MYND domain containing 14 | | |
| SET and MYND | ZMYND1; ZNFN3A1; FLJ21080; MGC104324; | 64754 | NP_073580.1 |
| domain containing 3 | bA74P14.1; SMYD3 | | |
| DOT1-like, histone | DOT1; KMT4; KIAA1814; DKFZp586P1823; | 84444 | NP_115871.1 |
| H3 methyltransferase | DOT1L | | |
| Nuclear SET domain- | WHS; NSD2; TRX5; MMSET; REIIBP; | 7468 | NP_001035889.1 |
| containing 2 | FLJ23286; KIAA1090; MGC176638; WHSC1; | | |
| | Wolf-Hirschhorn syndrome candidate 1 | | |
| Wolf-Hirschhorn | NSD3; pp14328; FLJ20353; MGC126766; | 54904 | NP_075447.1 |
| syndrome candidate 1- | MGC142029; DKFZp667H044; WHSC1L1; | | |
| like 1 | WHSC1L1 protein; | | |
| | Wolf-Hirschhorn syndrome candidate 1-like 1 | | |
| | protein | | |
| BMI1 polycomb ring | PCGF4; RNF51; MGC12685; BMI1; B lymphoma | 648 | NP_005171.4 |
| finger oncogene | Mo-MLV insertion region 1 homolog | | |
| PR domain containing | PFM11; MGC59730; PRDM14 | 63978 | NP_078780.1 |
| 14 | | | |
| PR domain containing | BLIMP1; PRDI-BF1; MGC118922; MGC118923; | 639 | NP_001189.2 |
| 1, with ZNF domain | MGC118924; MGC118925; PRDM1; | | |
| | OTTHUMP00000016918; | | |
| | PRDI-binding factor-1; | | |
| | PR-domain zinc finger protein 1; | | |
| | B-lymphocyte-induced maturation protein 1; | | |
| | positive regulatory domain I-binding factor 1; | | |
| | beta-interferon gene positive-regulatory domain I | | |
| | binding factor | | |
| myelodysplasia | PRDM3; MDS1-EVI1; MDS1; myelodysplasia | 4197 | NP_004982.1 |
| syndrome 1 | syndrome protein 1 | | |
| | myelodysplasia syndrome-associated protein 1 | | |
| PR domain containing 5 | PFM2; PRDM5 | 11107 | NP_061169.2 |
| PR domain containing | PFM9; PRDM12; OTTHUMP00000022367 | 59335 | NP_067632.2 |
| 12 | PR-domain containing protein 12 | | |
| | PR-domain zinc finger protein 12 | | |

TABLE 1B
Exemplary Demethylases

| | | GenBank | GenBank |
|---|---|---|---|
| | | GeneID | Acc. No. (amino |
| Name | Alternative names | No. | acid seq.) |
| Lysine-specific | AOF2; LSD1; BHC110; KIAA0601; RP1-184J9.1; | 23028 | NP_001009999.1 |
| histone | KDM1 | | |
| demethylase 1 | | | |
| lysine (K)- | FBL7; CXXC8; FBL11; FBXL11; JHDM1A; LILINA; | 22992 | NP_036440.1 |
| specific | FLJ00115; FLJ46431; KIAA1004; DKFZp434M1735; | | |
| demethylase 2A | KDM2A; F-box and leucine-rich repeat protein 11; | | |
| | F-box protein FBL11; | | |
| | jumonji C domain-containing histone demethylase 1A | | |
| lysine (K)- | CXXC2; Fb110; PCCX2; FBXL10; JHDM1B; KDM2B; | 84678 | NP_115979.3 |
| specific | F-box and leucine-rich repeat protein 10; | | |
| demethylase 2B | protein containing CXXC domain 2; | | |
| | jumonji C domain-containing histone demethylase 1B; | | |
| | JEMMA (Jumonji domain, EMSY-interactor, | | |
| | methyltransferase motif) protein | | |
| lysine (K)- | TSGA; JMJD1; JHDM2A; JHMD2A; JMJD1A; | 55818 | NP_001140160.1 |
| specific | KIAA0742; DKFZp686A24246; DKFZp686P07111; | | |
| demethylase 3A | KDM3A; jumonji domain containing 1A; | | |
| | OTTHUMP00000160707; | | |
| | testis-specific protein A | | |
| | jumonji domain containing 1; | | |
| | jumonji C domain-containing histone demethylase 2A | | |
| lysine (K)- | 5qNCA; C5orf7; JMJD1B; KIAA1082; KDM3B; | 51780 | NP_057688.2 |
| specific | jumonji domain containing 1B; | | |
| demethylase 3B | nuclear protein 5qNCA | | |
| lysine (K)- | JMJD2; JHDM3A; JMJD2A; KIAA0677; KDM4A; | 9682 | NP_055478.2 |
| specific | jumonji domain containing 2A; | | |
| demethylase 4A | OTTHUMP00000008810; | | |
| | jumonji domain containing 2; | | |
| | jumonji C domain-containing histone demethylase 3A | | |
| lysine (K)- | JMJD2B; FLJ44906; KIAA0876; KDM4B; jumonji | 23030 | NP_055830.1 |
| specific | domain containing 2B | | |
| demethylase 4B | | | |
| lysine (K)- | GASC1; JHDM3C; JMJD2C; FLJ25949; KIAA0780; | 23081 | NP_001140166.1 |
| specific | bA146B14.1; KDM4C; jumonji domain containing 2C; | | |
| demethylase 4C | OTTHUMP00000021052; | | |
| | OTTHUMP00000044461; | | |
| | gene amplified in squamous cell carcinoma 1; | | |
| | JmjC domain-containing histone demethylation protein | | |
| | 3C | | |
| lysine (K)- | JMJD2D; FLJ10251; MGC141909; KDM4D; jumonji | 55693 | NP_060509.2 |
| specific | domain containing 2D | | |
| demethylase 4D | | | |
| lysine (K)- | RBP2; RBBP2; JARID1A; KDM5A; | 5927 | NP_001036068.1 |
| specific | retinoblastoma binding protein 2; | | |
| demethylase 5A | retinoblastoma-binding protein 2; | | |
| | Jumonji, AT rich interactive domain 1A (RBP2-like); | | |
| | Jumonji, AT rich interactive domain 1A (RBBP2-like) | | |
| lysine (K)- | CT31; PUT1; PLU-1; JARID1B; FLJ10538; FLJ12459; | 10765 | NP_006609.3 |
| specific | FLJ12491; FLJ16281; FLJ23670; RBBP2H1A; KDM5B | | |
| demethylase 5B | | | |
| lysine (K)- | MRXJ; SMCX; MRXSJ; XE169; JARID1C; | 8242 | NP_004178.2 |
| specific | DXS1272E; KDM5C; jumonji, AT rich interactive | | |
| demethylase 5C | domain 1C; | | |
| | OTTHUMP00000023342; | | |
| | selected cDNA on X | | |
| | Smcy homolog, X-linked; | | |
| | Smcx homolog, X chromosome; | | |
| | JmjC domain-containing protein SMCX; | | |
| | Jumonji/ARID domain-containing protein 1C; | | |
| | Jumonji, AT rich interactive domain 1C (RBP2-like) | | |
| lysine (K)- | HY; HYA; SMCY; JARID1D; KIAA0234; KDM5D; | 8284 | NP_001140177.1 |
| specific | jumonji, AT rich interactive domain 1D; | | |
| demethylase 5D | Smcy homolog, Y-linked; | | |
| | SMC homolog, Y chromosome; | | |
| | Smcy homolog, Y chromosome; | | |
| | histocompatibility Y antigen; | | |
| | selected mouse cDNA on Y, human homolog of | | |
| | Jumonji, AT rich interactive domain 1D (RBP2-like) | | |
| lysine (K)- | UTX; MGC141941; bA386N14.2; DKFZp686A03225; | 7403 | NP_066963.2 |
| specific | KDM6A; ubiquitously transcribed tetratricopeptide | | |
| demethylase 6A | repeat, X chromosome | | |
| lysine (K)- | JMJD3; KIAA0346; KDM6B; lysine (K)-specific | 23135 | NP_001073893.1 |
| specific | demethylase 6B | | |
| demethylase 6B | jumonji domain containing 3, histone lysine demethylase | | |
| PHD finger | JHDM1F; MRXSSD; ZNF422; KIAA1111; | 23133 | NP_055922 |
| protein 8 | DKFZp686E0868; PHF8; PHD finger protein 8; | | |
| | OTTHUMP00000061869; | | |
| | jumonji C domain-containing histone demethylase 1F | | |
| jumonji domain | TRIP8; FLJ14374; KIAA1380; RP11-10C13.2; | 221037 | NP_004232.2 |
| containing 1C | DKFZp761F0118; JMJD1C; OTTHUMP00000060747; | | |
| | thyroid hormone receptor interactor 8; | | |
| | thyroid receptor interacting protein 8 | | |
| jumonji C | KIAA1718; JHDM1D | 80853 | NP_085150 |
| domain | | | |
| containing | | | |
| histone | | | |
| demethylase 1 | | | |
| homolog D | | | |
| PHD finger | GRC5; JHDM1E; KIAA0662; MGC176680; PHF2; PHD | 5253 | NP_005383 |
| protein 2 | finger protein 2; | | |
| | jumonji C domain-containing histone demethylase 1E | | |
| amine oxidase | C6orf193; FLJ33898; FLJ34109; FLJ43328; bA204B7.3; | 221656 | NP_694587.3 |
| (flavin | dJ298J15.2; DKFZp686I0412; AOF1; | | |
| containing) | OTTHUMP00000016075; | | |
| domain 1 | OTTHUMP00000016077; | | |
| | OTTHUMP00000039336; | | |
| | amine oxidase, flavin containing 1 | | |

### Substrates of Histone Methyl Modifying Enzymes

Any substrate for histone methyl-modifying activity can be used according to methods of the present disclosure. In some embodiments, a substrate comprises a full length histone polypeptide or portion thereof. In some embodiments, a substrate comprises a nucleosome. In some embodiments, a substrate comprises an oligonucleosome. In some embodiments, a substrate comprises a reconstituted nucleosome. In some embodiments, a substrate comprises a nucleosome purified from a cell (see, e.g., Ausio and van Holde, Biochem. 25:1421-1428, 1986; and Fang et al., Meth. Enzymol. 377:213-226, 2003). In some embodiments, a substrate comprises chromatin. Histones used as substrates can include histones from one or more species.

In some embodiments, a substrate comprises a peptide (e.g., a histone peptide). For example, analysis of activity demethylase enzyme may utilize a histone peptide (e.g., as shown in Examples herein).

### Stimulating Agents

Stimulating agents provided herein comprise peptides. In some embodiments, stimulating agents are 4-60 amino acids in length. For example, a stimulating agent can include 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a stimulating agent includes 4-60 amino acids of a histone polypeptide (e.g., an H3, H4, H1, H2A, or H2B histone polypeptide). In some embodiments, a stimulating agent comprises 4-60 amino acids from the N-terminus of a histone polypeptide. In some embodiments, a stimulating agent comprises a methylated peptide.

Methylation of a stimulating agent can include mono-, di-, and/or tri-methylation. A stimulating agent can include methylation of one or more residues (e.g., one or more lysine residues).

In some embodiments, a stimulating agent comprises at least four amino acids from an N-terminal region (e.g., an N-terminal region comprising the N-terminal 60 amino acids) of a histone polypeptide, wherein the agent comprises a methylated lysine. In some embodiments, a stimulating agent has a sequence derived from a natural methylase substrate, and is methylated at a position in which the natural methylase substrate is methylated. For example, enzymes methylate histone H3 on lysines 4, 9, 27, 36, and 79 (H3K4, H3K9, H3K27, H3K36, and H3K79). H4 is methylated on lysine 20 (H4K20). Thus, for example, a stimulating agent can include a methylated histone peptide comprising one or more of H3K4 (“H3K4” refers to lysine 4 of an H3 histone polypeptide, wherein the numbering corresponds to position of lysine 4 in a wild type H3 histone polypeptide sequence), H3K9, H3K27, H3K36, H3K79, or H4K20.

In some embodiments, a stimulating agent comprises at least four amino acids of the following sequence of a histone H3 polypeptide, wherein the agent comprises a methylated lysine: ARTKQTARKSTGGKAPRKQLATKAARKSAPATGESKKPHRYRPGTAALREIRRYQKST EL (SEQ ID NO:1). A stimulating agent can also include a peptide having one or more amino acid substitutions relative to SEQ ID NO:1 (e.g, substitutions at one, two, three, four, or five positions) other than at the methylated lysine residue. In some embodiments, a substitution is a substitution found in a histone H3 sequence of a non-human species. In some embodiments, a stimulating agent includes at least four amino acids of SEQ ID NO:1 having an alanine to serine substitution at residue 31 (A31S).

In some embodiments, a stimulating agent comprises at least four amino acids of SEQ ID NO:1, wherein the sequence includes one or more of H3 K4, K9, K27, K36, or K79. For example, a stimulating agent comprising H3K4 can include one of the following sequences: ARTK (SEQ ID NO:6); ARTKQ (SEQ ID NO:7); ARTKQT (SEQ ID NO:8); ARTKQTA (SEQ ID NO:9); ARTKQTAR (SEQ ID NO:10); ARTKQTARK (SEQ ID NO:11); RTKQ (SEQ ID NO:12); RTKQT (SEQ ID NO:13); TKQT (SEQ ID NO:14); KQTA (SEQ ID NO:15).

In some embodiments, a stimulating agent comprises an H3K27 sequence as follows: RKQLATKAAR(KMe3)SAPATGGVKKP (SEQ ID NO:16). (“me3” denotes the presence of trimethylation on a lysine residue.)

In some embodiments, a stimulating agent comprises an H3K9 sequence as follows: ARTKQTAR[Kme3]STGGKAPRKQLA (SEQ ID NO:17).

In some embodiments, a stimulating agent comprises at least four amino acids of the following sequence of a histone H4 polypeptide, wherein the agent comprises a methylated lysine: SGRGKGGKGLGKGGAKRHRKVLRDNIQGITKPAIRRLARRGGVKRISGLIYEETRGVLK V (SEQ ID NO:2). A stimulating agent can also include a peptide having one or more amino acid substitutions relative to SEQ ID NO:2 (e.g, substitutions at one, two, three, four, or five positions) other than at the methylated lysine residue. In some embodiments, a substitution is a substitution found in a histone H4 sequence of a non-human species (e.g., a V21A or V21I substitution).

In some embodiments, a stimulating agent comprises an H4K20 sequence as follows: LGKGGAKRHR[Kme3]VLRDNIQGIT (SEQ ID NO:18).

In some embodiments, a stimulating agent comprises at least four amino acids of the following sequence of a histone H1.4 (H1e) polypeptide, wherein the agent comprises a methylated lysine: SETAPAAPAAPAPAEKTPVKKKARKSAGAAKRKASGPPVSELITKAVAASKERSGVSLA A (SEQ ID NO:3). A stimulating agent can also include a peptide having one or more amino acid substitutions relative to SEQ ID NO:3 (e.g, substitutions at one, two, three, four, or five positions) other than at the methylated lysine residue. In some embodiments, a substitution is a substitution found in a histone H1e sequence of a non-human species. In some embodiments, a stimulating agent comprises an H1.4K26 sequence as follows: VKKKAR[Kme2]SAGAAKRKASG (SEQ ID NO:19).

In some embodiments, a stimulating agent comprises at least four amino acids of the following sequence of a histone H1e polypeptide, wherein the agent comprises a methylated lysine: SETAPAAPAAPAPAEKTPVKKKARKAAGGAKRKTSGPPVSELITKAVAASKERSGVSLA A (SEQ ID NO:4). A stimulating agent can also include a peptide having one or more amino acid substitutions relative to SEQ ID NO:4 (e.g, substitutions at one, two, three, four, or five positions) other than at the methylated lysine residue. In some embodiments, a substitution is a substitution found in a histone H1e sequence of a non-murine species.

Additional stimulating agents are described in the Examples herein.

Peptides can be produced by chemical synthesis or recombinant expression. Peptides can be methylated by any available means (e.g., by chemical or enzymatic methods).

Stimulating agents can include modifications in addition (or alternative) to methylation, such as acetylation, phosphorylation, hydroxylation, glycosylation, sulfation, or lipidation.

A stimulating agent can be labeled, e.g., at its N-terminus, C-terminus, or internally. A label can be coupled to a stimulating agent directly or via a linker or spacer. Useful labels include radioactive moieties, enzymes, and fluorescent moieties. In some embodiments, a stimulating agent is labeled with biotin.

### Assays

Test Compounds

The present disclosure provides assays for screening for a test compound, or more typically, a library or collection of test compounds, to evaluate an effect of the test compound on activity of a histone methyl modifying enzyme in vitro (e.g., on a methylase or a demethylase).

A test compound can be the only substance assayed by a method described herein. Alternatively, a collection of test compounds can be assayed either consecutively or concurrently by methods described herein. Members of a collection of test compounds can be evaluated individually or in a pool, e.g., using a split-and-pool method.

In one embodiment, high throughput screening methods are used to screen a combinatorial chemical or peptide library, or other collection, containing a large number of potential HMME modulatory compounds (test compounds). Such “combinatorial chemical libraries” are then screened in one or more assays to identify those library members (particular chemical species or subclasses) that display a desired characteristic activity. Compounds thus identified can serve as conventional “lead compounds” or can themselves be used as potential or actual modulators (e.g., as therapeutics).

A combinatorial chemical library typically includes a collection of diverse chemical compounds, for example, generated by either chemical synthesis or biological synthesis, by combining a number of chemical “building blocks” such as reagents. For example, a linear combinatorial chemical library such as a polypeptide library may be formed by combining a set of chemical building blocks (amino acids), e.g., in particular specified arrangements or in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., scaffold or framework).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Test compounds can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; synthetic library methods using affinity chromatography selection, or any other source, including assemblage of sets of compounds having a structure and/or suspected activity of interest. Biological libraries include libraries of nucleic acids and libraries of proteins. Some nucleic acid libraries provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Assay Methods

Any assay herein, e.g., an in vitro assay, can be performed individually, e.g., just with the test compound, or with other appropriate controls. A “control” reaction is typically a reaction identical to a test reaction except for the change of a single parameter (or, in some cases, a small number of parameters). For example, a control reaction may be a parallel assay without a test compound, or a other parallel assay without one or more other reaction components, e.g., without a target or without a substrate. In some embodiments, it is possible to compare assay results to a reference, e.g., a reference value, e.g., obtained from the literature, a prior assay, and so forth. Appropriate correlations and art known statistical methods can be used to evaluate an assay result.

Once a compound is identified as having a desired effect (e.g., modulation of activity of a histone methyl modifying enzyme), production quantities of the compound can be synthesized, e.g., producing at least 50 mg, 500 mg, 5 g, or 500 g of the compound. The compound can be formulated, e.g., for administration to a subject, and may also be administered to the subject.

Activity of histone methyl modifying enzymes can be evaluated in an in vitro system. The effect of a test compound can be evaluated, for example, by measuring methylation of a substrate in the presence of a stimulating agent at the beginning of a time course, and then comparing such levels after a predetermined time (e.g., 0.1, 0.25, 0.5, 1, 1.5, 2, 2.5, 3, or more hours) in a reaction that includes the test compound and in a parallel control reaction that does not include the test compound. This is one example of a method for determining the effect of a test compound on enzyme activity in vitro using a stimulating agent as provided by the present disclosure.

In general, an assay involves preparing a reaction mixture of a histone methyl modifying enzyme, a substrate, a stimulating agent, and one or more test compounds under conditions and for a time sufficient to allow components to interact. Methylation can be evaluated directly or indirectly.

In some embodiments, a component of an assay reaction mixture (e.g., a substrate) is anchored onto a solid phase. A component anchored on the solid phase can be detected at the end of a reaction, e.g., a methylase reaction. Any vessel suitable reactants can be used. Examples of suitable vessels include microtiter plates, test tubes, and micro-centrifuge tubes.

Activity of methyl modifying enzymes can be evaluated by any available means. In some embodiments, a methylation state of a substrate is evaluated by mass spectrometric analysis of a substrate. In some embodiments, methylation of a substrate is evaluated with an antibody specific for a methylated or demethylated substrate. Such antibodies are commercially available (e.g., from Upstate Group, NY, or Abcam Ltd., UK). Suitable immunoassay techniques for detecting methylation state of a substrate include immunoblotting, ELISA, and immunoprecipitation.

Methylation reactions can be carried out in the presence of a labeled methyl donor (e.g., a S-adenosyl-[methyl-¹⁴C]-L-methionine, or 5-adenosyl-[methyl-³H]-L-methionine), allowing detection of label into a methylase substrate, or release of label from a demethylase substrate.

In some embodiments, activity of a methyl modifying enzyme is evaluated using fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on a ‘donor’ (e.g., a DNA molecule of a nucleosome) is selected such that its emitted fluorescent energy will be absorbed by a fluorescent label on an ‘acceptor’ (e.g., an antibody specific for a histone methyl modification of interest), which in turn is able to fluoresce due to the absorbed energy. A reaction can be carried out using an unlabelled substrate, and histone modification is determined by detecting antibody binding using a fluorimeter (see, U.S. Pat. Pub. 2008/0070257).

In some embodiments, demethylation is evaluated by direct or indirect detection of release of a reaction product such as formaldehyde and/or succinate. In some embodiments, release of formaldehyde is detected. Release of formaldehyde can be detected using a formaldehyde dehydrogenase assay in which formaldehyde dehydrogenase converts released formaldehyde to formic acid using NAD⁺ as electron acceptor. Reduction of NAD⁺ can be detected spectrophotometrically (Lizcano et al., Anal. Biochem. 286:75-79, 2000). In some embodiments, release of formaldehyde is detected by converting formaldehyde to 3,5-diacethyl-1,4-dihydrolutidine (DDL) and detecting the DDL, for example, by detecting radiolabeled DDL (e.g., ³H-DDL). A substrate can be labeled so that a labeled reaction product is released (e.g., formaldehyde and/or succinate) by a demethylation reaction. In some embodiments, a substrate is methylated with ³H-SAM (S-adenosylmethionine), demethylation of which releases ³H-formaldehyde, which can detected directly, or which can be converted to ³H-DDL, which is detected. Methods of detecting reaction products such as formaldehyde and/or succinate include mass spectrometry, gas chromatography, liquid chromatography, immunoassay, electrophoresis, and the like, and combinations thereof. Demethylase assays are also described in Shi et al., Cell 119:941-953, 2004.

An alternative means for detecting demethylase activity employs analysis of release of radioactive carbon dioxide. See, e.g., Pappalardi et al., Biochem. 47(43):11165-11167, 2008, and Supporting Information, which describes use of a radioactive assay in which capture of ¹⁴CO₂ is captured and detected following release from α-[1-¹⁴C]-ketoglutaric acid coupled to hydroxylation reactions. Such methods can also be employed for detection of demethylation.

Detection of enzyme activity can include use of fluorescent, radioactive, scintillant, or other type of reagents. In some embodiments, a scintillation proximity assay is used for evaluating enzyme activity. Such assays can involve use of an immobilized scintillant (e.g., immobilized on a bead or microplate) and a radioactive methyl donor. In some embodiments, a scintillation proximity assay employs scintillant-coated microplates such as FlashPlates® (Perkin Elmer).

In some embodiments, components of an assay reaction mixture are conjugated to biotin and streptavidin. Biotinylated components (e.g., biotinylated substrate or biotinylated stimulating agent) can be prepared, e.g., using biotin-NHS (N-hydroxy-succinimide) according to known techniques (e.g., biotinylation kit, Pierce Chemicals, Rockford, Ill.). Biotinylated components can be captured using streptavidin-coated beads or immobilized in the wells of streptavidin-coated plates (Pierce Chemical).

As would be appreciated by those of skill in the art, assays can employ any of a number of standard techniques for preparation and/or analysis of enzymatic activity, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect activity of histone methyl modifying enzymes.

Test compounds identified as enzyme modulators using in vitro assays described herein can be further evaluated in an animal model. An animal model can include a mammal (e.g., a mouse, rat, primate, or other non-human), or other organism (e.g., Xenopus, zebrafish, or an invertebrate such as a fly or nematode). In some cases, an animal model uses a transgenic organism, e.g., an organism which includes a heterologous histone methyl modifying enzyme. A test compound can be administered to an animal once or as a regimen (regular or irregular). A parameter of the animal is then evaluated, e.g., a parameter of a pathway regulated by the histone methyl modifying enzyme, such as cell proliferation or differentiation. Test compounds that are indicated as of interest result in a change in the parameter relative to a reference, e.g., a parameter of a control animal. Other parameters (e.g., related to toxicity, clearance, and pharmacokinetics) can also be evaluated.

In some embodiment, a test compound is evaluated using an animal that has a particular disorder, e.g., a cell proliferative disorder, or using an animal that is otherwise sensitized to developing a particular disorder, e.g., a cell proliferative disorder.

Screening assays or any information described herein can be evaluated using standard statistical methods. For example, data can be expressed as mean±SEM. Differences can be analyzed by ANOVA; significance can be assessed at the 95% and 99% significance levels by the Fisher PLSD statistical test or by the paired 2-tailed t test. Data involving more than 2 repeated measures can be assessed by repeated-measures ANOVA. Non-normally distributed data can be compared using the Mann-Whitney U test.

### EXEMPLIFICATION

### Example 1

### High Throughput Demethylase Assays

High throughput demethylase assays can be performed in the presence of a stimulating agent according to the following exemplary protocol.

Materials and Reagents:
1. E. coli BL21 (DE3) expressed Human GASC1 (aa1-350) enzyme (In House prep)
2. H3K9me3 peptide (New England Peptide, Gardner, Mass.)
3. TrisHCl (pH 7.4, at room temperature) (Cat# 4109-07, J. T. Baker Phillipsburg, N.J.)
4. α-Ketoglutaric acid, sodium salt (Cat# K2010, Sigma Aldrich, St. Louis, Mo.)
5. (+)-Sodium L-ascorbate (Cat# A4034, Sigma Aldrich, St. Louis, Mo.)
6. Ammonium iron (II) sulfate hexahydrate (Cat# F1543, Sigma Aldrich, St. Louis, Mo.)
7. Glycerol (Cat# BP229, Fisher Scientific, Fair Lawn, N.J.)
8. Triton X-100 (Cat# T9284, Sigma Aldrich, St. Louis, Mo.)
9. Reaction Stop Mix—1N Hydrochloric acid (Cat# BDH3202-1, VWR, West Chester, Pa.)
10. Multidrop Combi (Cat# 5840300, Thermo Fisher Scientific, Waltham, Mass.)

General Procedure for Use with Multidrop:
1. Prepare fresh co-factor stocks each day that reactions are to be run
2. Prepare the Reaction Mix with ascorbate (135 ml), keep on ice:
   11.25 ml 1M TrisHCl, pH 7.4
   2.25 ml 100 mM Ascorbate (made fresh)
   22.5 ml 50% Glycerol 1.125 ml 2% Triton X-100
   93.78 ml deionized water
3. Prepare the Initiation Mix (100 ml), keep at room temperature
   250 μl 20 mM Ammonium iron (II) sulfate hexahydrate
   125 μl 10 mM α-Ketoglutaric acid, sodium salt
   99.625 ml deionized water
4. Rinse the Multidrop by priming with 50 ml Milli-Q water
5. Rinse the Multidrop by priming with 10 ml 1N HCl
6. Using the Multidrop, pre-quench any MIN control wells, dispense 25 μl of 1N HCl to each MIN well on the Thermo matrix 384 polypro 4314 plate.
7. Split the above Reaction Mix with ascorbate solution into 2 bottles, keep both on ice. Use one 65 ml aliquot of the Reaction Mix with ascorbate to wash the Multidrop machine prior to plating the reaction mix.
8. Rinse the Multidrop by priming with 100 ml Milli-Q water
9. Rinse the Multidrop by priming with 50 ml chilled Reaction Mix with ascorbate
10. To the other 65 mL aliquot of the Reaction Mix with ascorbate add 1.0 ml of 2.0 ug/ul GASC1 enzyme and 217 ul of 10 mM H3K9me3 peptide just prior to dispensing the reaction mix to the plate. 65 ml of Reaction mix with ascorbate, enzyme and H3K9me3 peptide yields ˜4250 reactions.
11. Using the Multidrop dispense 15 μl of Reaction Mix with ascorbate, enzyme, and peptide to each well on the plate. Once dispensing is complete, shake the plate for 5 seconds. Repeat with the next plate every 20 seconds. Be sure to make note of the order in which the plates are run through the Multidrop as this needs to be the same order in which the plates are initiated and quenched.
12. Rinse the Multidrop by priming with 50 ml Milli-Q water.
13. Rinse the Multidrop by priming with 30 ml Initiation Mix.
14. Start a timer at the initiation of Initiation Mix dispense. Using the Multidrop dispense 10 μl of Initiation Mix to each well on the plate. Once dispensing is complete, shake the plate for 5 seconds. Repeat with the next plate every 20 seconds. Be sure to make note of the order in which the plates are run through the Multidrop as this needs to be the same order in which the plates are quenched.
15. Let the initiated reaction run for 45 minutes.
16. Rinse the Multidrop by priming with 50 ml Milli-Q water.
17. Rinse the Multidrop by priming with 10 ml 1N HCl
18. Once the reaction has completed, use the Multidrop to quench all of the wells except for the MIN control well previously quenched, dispense 25 uL of 1N HCl to each well on the plate. Be sure to quench each plate in the same order as they were initiated and in 20 second intervals.
19. Heat seal the plate and store at −80° C.

Demethylation can be analyzed by mass spectrometry. The removal of a methyl group from a substrate such as H3K9me3 results in the loss of 15 mass units, to produce H3K9me2. Further demethylations of H3K9me2 yield losses of 14 mass units each. The difference in mass allows for quantitative determination of concentrations of analytes in complex mixtures.

### Example 2

### Stimulation of KDM4/JMJD2 Demethylase Family Members

JMJD2A, JMJD2B and JMJD2C/GASC1 proteins contain double PHD and Tudor domains in its C-terminus. The double Tudor domain of JMJD2A has been shown to specifically recognize H3K4me3 and H4K20me3 marks on histone H3 and H4 tails. It is likely that the double Tudor domains of JMJD2B and JMJD2C/GASC1 preserve the same binding specificity. All JMJD2 family members have been shown to be H3K9me3 demethylases and JMJD2A and JMJD2B has also been shown to catalyze H3K36me3 demethylation in vitro.

In a peptide demethylation reaction, JMJD2C/GASC1 can utilize H3K9me3 and H3K36me3 peptide as substrates and produce di-methylated lysine preferentially. The enzyme can also catalyze di to mono demethylation, but to a less robust extent. Since the H3 lysine 4 residue localizes in the same H3 polypeptide of H3 lysine 9 and H3 lysine 36, it was examined whether inclusion of an H3K4me3 mark on the peptide substrates stimulates JMJD2C/GASC1 activity by promoting enzyme and substrate recognition.

Experiments: Flag tagged full length JMJD2C/GASC1 was purified from insect cells. The peptide substrates contain the amino acid sequence of 1-21 residues of Histone H3, and trimethylation groups were introduced into the peptide substrates by chemical synthesis.

The following peptide was used as a substrate of JMJD2C/GASC1 enzyme activity:

H3 (1-21) K9me3 peptide: H2N-ARTKQTAR(KMe3)STGGKAPRKQLA-OH (SEQ ID NO:20)

The following peptide was used as a candidate stimulating agent of JMJD2C/GACS1 enzyme activity:

H3 (1-21) K4me3K9me3 peptide: H2N-ART(KMe3)QTAR(KMe3)STGGKAPRKQLA-OH (SEQ ID NO:21)

Assays were performed as described in Example 1.

Result: A stimulating effect on JMJD2C/GASC1 demethylase activity was observed in the presence of a peptide having a trimethyl group on H3 lysine 4.

### Example 3

### Stimulation of KDM5/JARID1 Demethylase Family Members

JARID1A-D proteins contain multiple PHD domains, and the N-terminus PHD domain of JARID1C/SMCX has been shown to specifically recognize H3K9me3 mark. It is likely that the corresponding PHD domains of the other family members preserve the same binding specificity. JARID1 family enzymes have been shown to be H3K4me3 demethylases in vitro.

In a peptide demethylation reaction, JARID1C/SMCX can utilize H3K4me3 peptide as substrate and produce di-methylated lysine preferentially. The enzyme can also catalyze di to mono demethylation, but to a less robust extent. Since the H3 lysine 9 residue localizes in the same H3 polypeptide of H3 lysine 4, it was examined whether the presence of an H3K9me3 mark on the peptide substrates stimulates JARID1C/SMCX activity by promoting enzyme and substrate recognition.

Experiments: Flag tagged full length JARID1A/SMCX was purified from insect cells. The peptide substrates contain the amino acid sequence of 1-21 residues of Histone H3, and trimethylation groups were introduced into the peptide substrates by chemical synthesis.

The following peptide was used as a substrate of JARID1C/SMCX enzyme activity:

H3 1-21H3K4me3 peptide: H2N-ART(KMe3)QTARKSTGGKAPRKQLA-OH (SEQ ID NO:22)

The following peptide was used as a candidate stimulating agent:

H3 1-21H3K4me3K9me3 peptide: H2N-ART(KMe3)QTAR(KMe3)STGGKAPRKQLA-OH (SEQ ID NO:23)

Demethylation reactions were performed as described in Example 1.

Result: A stimulating effect on JARID1C/SMCX activity was observed in the presence of a peptide having a trimethyl group on H3 lysine-9.

### Example 4

### Stimulation of PHF2, PHF8 and KIAA1718 Demethylase Family Members

PHF2, PHF8 and KIAA1718 proteins contain one N-terminus PHD domain. The N-terminus PHD domains are likely to bind H3K4me3 mark in histone H3 due to sequence similarity to known PHD domain recognizing H3K4me3 mark, such as BPTF and ING2. PHF8 and KIAA1718 has been shown to be H3K9me2 and H3K27me2 demethylases in vitro, respectively (unpublished observations).

In a peptide demethylation reaction, PHF8 can utilize H3K9meme2 peptide as substrate and produce mono-methylated lysine preferentially. The enzyme can also catalyze mono to zero demethylation, but to a less robust extent. Since the H3 lysine 4 residue localizes in the same H3 polypeptide of H3 lysine 9, it was examined whether inclusion of an H3K4me3 mark on the peptide substrates stimulates PHF8 activity by promoting enzyme and substrate recognition.

Experiments: Flag tagged full length PHF8 was purified from insect cells. The peptide substrates contain the amino acid sequence of 1-21 residues of Histone H3, and trimethylation groups were introduced into the peptide substrates by chemical synthesis.

The following peptide was used as a substrate of PHF2/PHF8 enzyme activity:

H3 1-21H3K9me2 peptide: H2N-ARTKQTAR(KMe2)STGGKAPRKQLA-OH (SEQ ID NO:24)

The following peptide was used as a candidate stimulating agent:

H3 1-21H3K4me3K9me2 peptide: H2N-ART(KMe3)QTAR(KMe2)STGGKAPRKQLA-OH (SEQ ID NO:25)

Demethylation reactions were performed as described in Example 1.

Result: A stimulating effect on PHF2/PHF8 demethylase activity was observed in the presence of a peptide having a trimethyl group on H3 lysine-9.

### Example 5

### High Throughput Methylase Assays

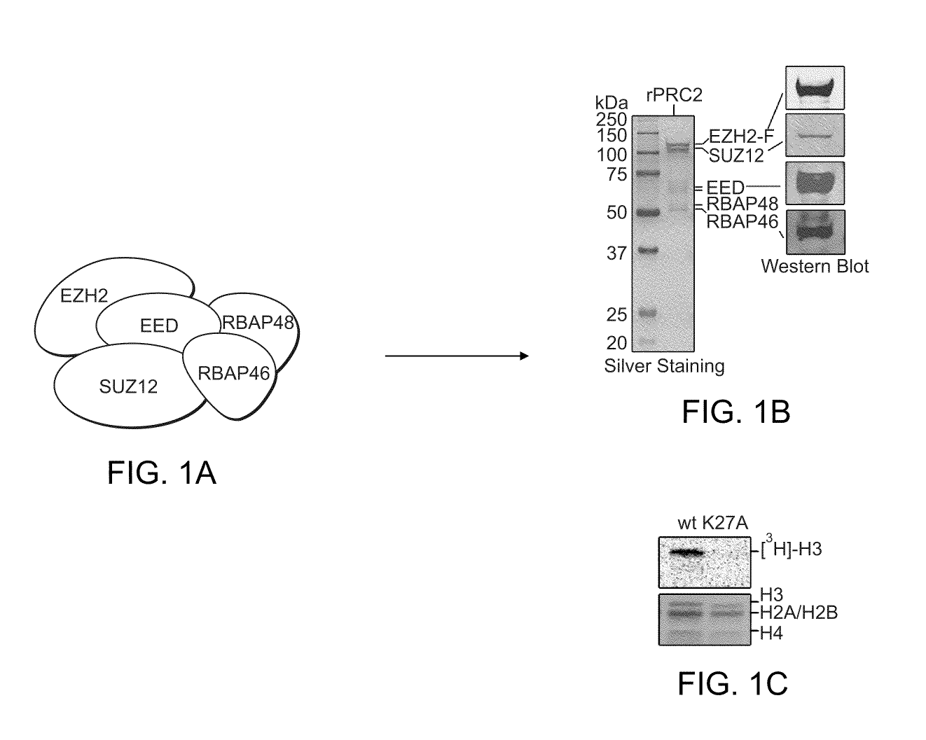

Polycomb repressive complex 2 (PRC2) is a multisubunit methylase complex that includes EZH2 (Enhancer of Zeste Homolog 2), EED, SUZ12, Rbap46, and Rbap48 subunits. Reconstituted PRC2 complexes (MW=600 kDa) were used in vitro assays to determine methylase activity in the presence of novel stimulating agents. A schematic depiction of a reconstituted PRC2 complex is shown in FIG. 1A. Silver staining and Western blot analysis are shown in FIG. 1B. Methylation of wt and K27A H3 substrates are shown in FIG. 1C.

### High Throughput Methylase Assay

The following reaction mix was used for high throughput methylase assays:

30 μl Reaction Mix

| | | | |
|---|---|---|---|
| 6.0 | μl | 5x HMT buffer | |
| 0.45 | μl | DTT 0.2M = | 3 mM |
| 1.0 | μl | ³H-SAM (Perkin Elmer, 0.55 μCi/μl) = | 0.24 μM |
| 1.0 | μl | H3K27me3 peptide [0.1 mg/ml] = | 1.24 μM |
| 1.0 | μl | rPRC2 [0.319 mg/ml] = | 17.7 nM |
| 0.11 | μl | Bio/Avi-oligonucleosomes [1.0 mg/ml] = | 1.5 nM |
| 0.24 | μl | DMSO/compounds = | 0.79% |
| 9.80 | μl | | |
| 20.2 | μl | water | |

Reaction mixtures were incubated for 60 min. at 30° C. in 384-well Black and White Microplates, Polystyrene (Greiner Bio-One Black FLUOTRAC 200 Medium Binding Nonsterile Greiner Bio-One No. 781096, VWR Catalog # 82051-294). For detection, Streptavidin FlashPlate HTS PLUS were used (High Capacity, 384 well, Perkin Elmer product # SMP410001PK).

A workflow used for high throughput assays was as follows:
1. Preparation of assay plates using the ECHO® (Labcyte) (240 nl of DMSO or compound per well)
2. Preparation of Mix I+II; I: 1202×15 μl; II: 1202×15.0 μl (dead volume ˜50 reactions)
   Mix I
   6.0 μl 5× HMT buffer
   0.45 μl DTT 0.2M
   1.0 μl rPRC2
   1.0 μl H3K27me3 peptide
   0.11 μl Bio/Avi-oligonuc. 1:10
   6.44 μl water
   15.0 μl
   Mix II
   1.0 μl ³H-SAM
   14.0 μl water
   15.0 μl
3. Adding Mix I to assay plate using the Multidrop (15 μl per well)
4. Adding Mix II to assay plate using the Multidrop (15 μl per well)
5. Mix content of wells in the assay plate
6. Spin plate for 1 min at 400 RPM
7. Incubate assay plate for 60 min at 30° C.
8. Quench reaction by adding 30 μl of SAH [1.25 mM] to each well (mix); final conc.=568 μM
9. Spin plate for 1 min at 400 RPM
10. Transfer reactions to FLASHplate using the BRAVO
11. Spin plate for 1 min at 400 RPM
12. Incubate FLASHplate at RT for 20 min under agitation
13. Remove liquid from all wells using the plate washer
14. Wash FLASHplate 2× with 60 μl wash buffer (20 mM Tris, pH8, 200 mM NaCl, 0.5% NP40) using the Multidrop
15. Remove wash buffer using plate washer
16. Analyze FLASHplate using the Topcount (2 min per well)

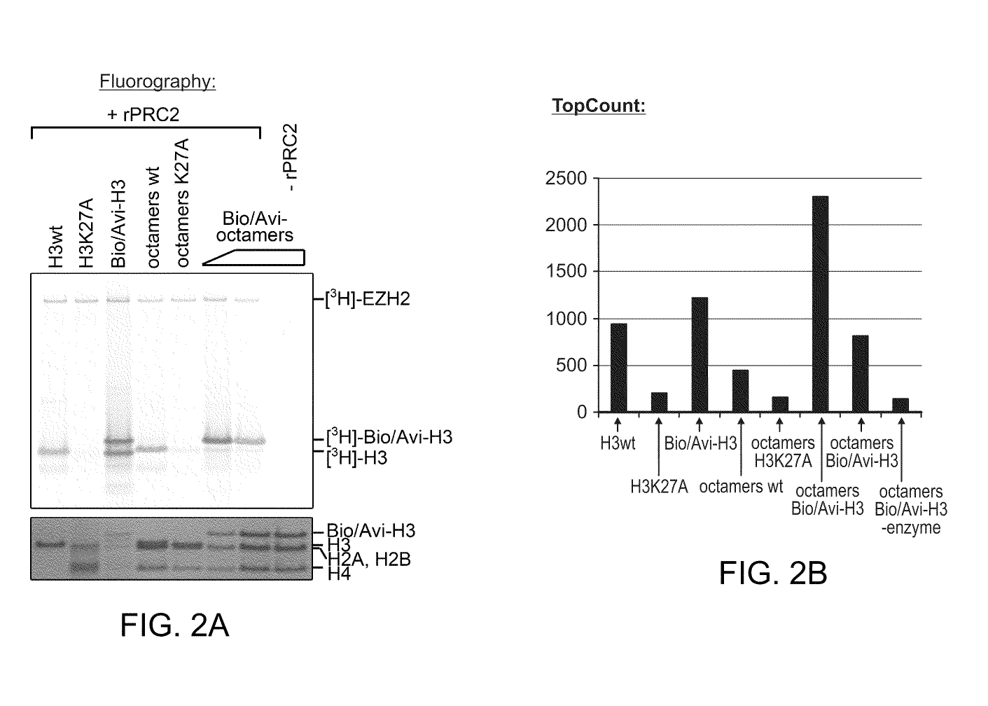

Methylase assays were performed in the presence and absence of rPRC2 using the following substrates: wt H3, H3K27A, Bio/Avi-H3, wt octamers, K27A octamers, and different concentrations of Bio/Avi-octamers. H3 methylation was analyzed by fluorography and TopCount, which is a scintillation proximity assay (SPA). The results for assays using these substrates are shown in FIGS. 2A and 2B. The greatest degree of methylation was observed with the lower concentration of Bio/Avi-octamers, followed by Bio/Avi-H3, H3 wt, and higher concentrations of Bio/Avi-H3 octamers.

### Oligonucleosome Titration

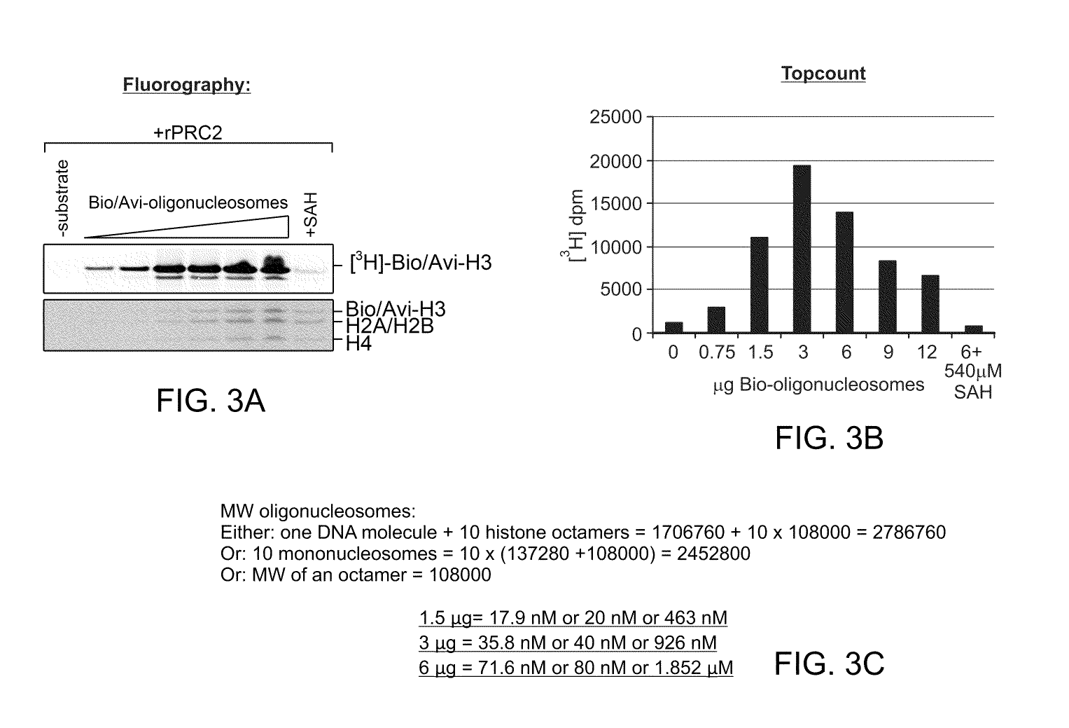

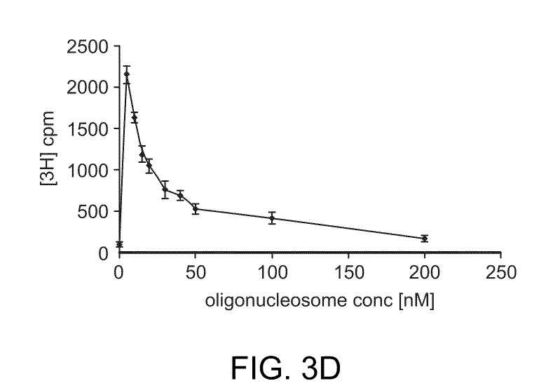

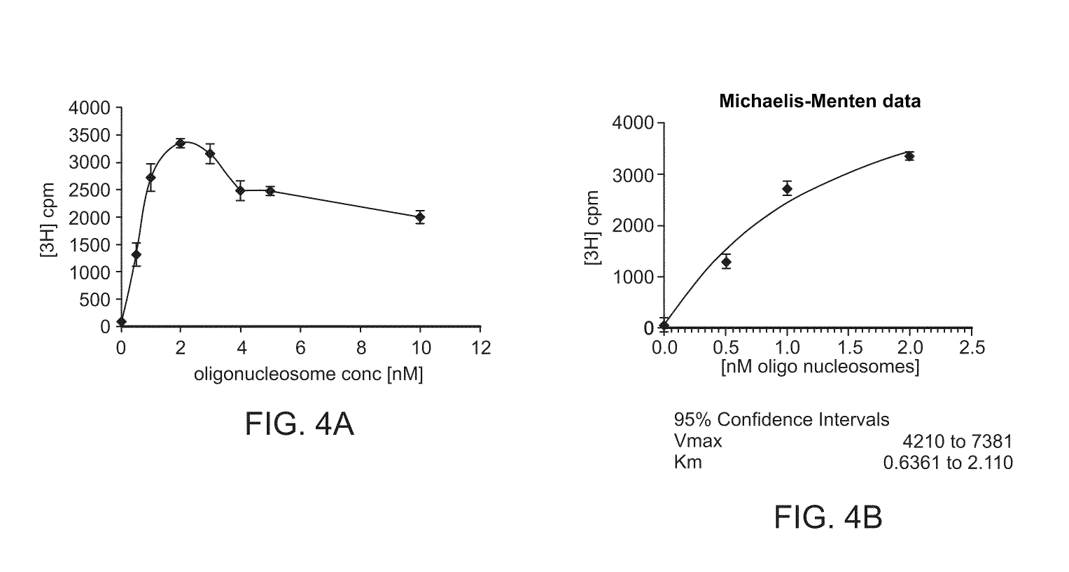

Methylase assays were performed as described above, using Bio/Avi-oligonucleosomes at increasing concentrations. The results are shown in FIGS. 3A, 3B, and 3D. Km measurements of oligonucleosome methylase activity are shown in FIGS. 4A and 4B.

### Example 6

### Stimulation of rPRC2 Methylase Family Members

Stimulation of rPRC2 methylase activity was determined in the presence of unmodified H3, H3K4me3, H3K9me3, H3K27me3, H3K36me3, H3K79me3, H4K20me3, and H1.4K26me3. Reactions had the following components:

Enzyme: 12.15 nM

[³H]-SAM: 0.24 μM

DTT: 3 mM

Oligonucleosomes: 14.95 nM

Histone peptides: ˜1.86 μM

Peptides used in stimulation assays included the following:

(SEQ ID NO: 26)
H3K27me3: H2N-RKQLATKAAR(KMe3)SAPATGGVKKP-COOH
(SEQ ID NO: 27)
H3K9me3-Bio ARTKQTAR[Kme3]STGGKAPRKQLA(-Biotin)
(SEQ ID NO: 28)
H4K20me3-Bio LGKGGAKRHR[Kme3]VLRDNIQGIT(-Biotin)
(SEQ ID NO: 29)
H1.4K26me3-Bio VKKKAR[Kme2]SAGAAKRKASG(-Biotin)

Reactions were incubated for 45 min. at 30° C. Reactions were stopped by the addition of 450 μM SAH (final concentration; total vol.=60 μl). Reactions were incubated on FLASHplates for 45 min. and washed twice with 60 μl wash buffer.

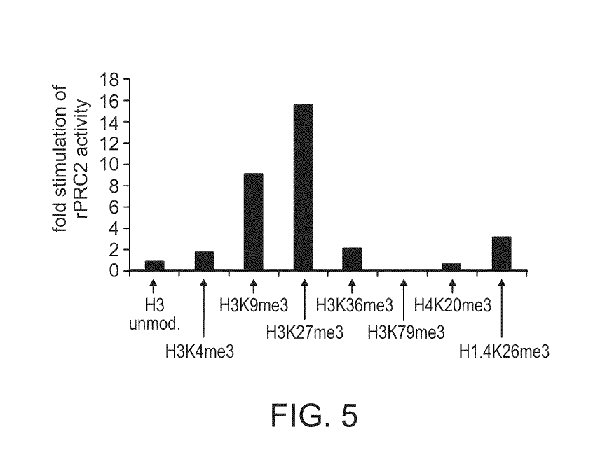

As shown in FIG. 5, H3K27me3 stimulated rPRC2 methylase activity toward oligonucleosomes over 15-fold (relative to unmodified H3). H3K9me3 stimulated activity over 9-fold. Stimulation by H3K4me3, H3K36me3, and H1.4K26me3 was also observed.

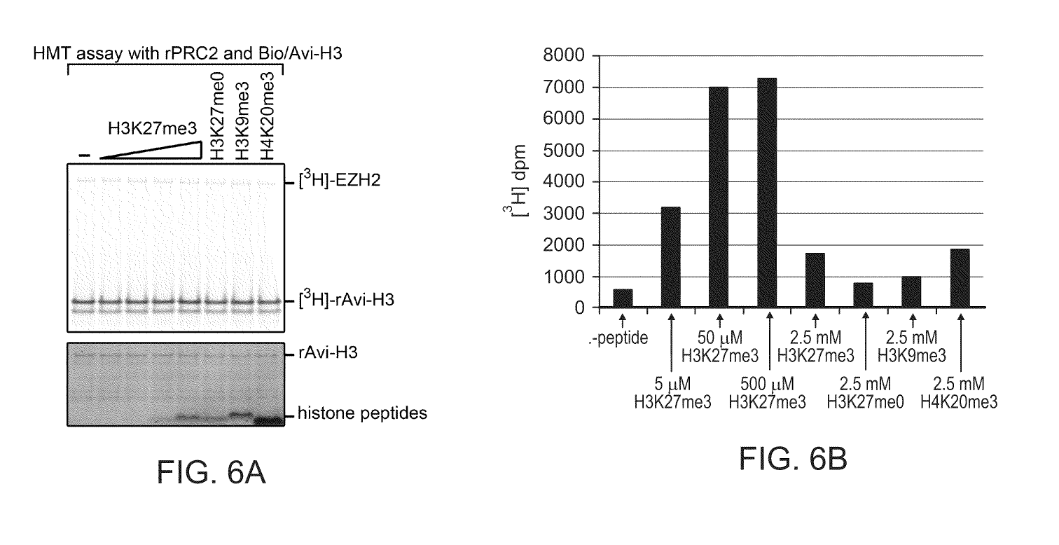

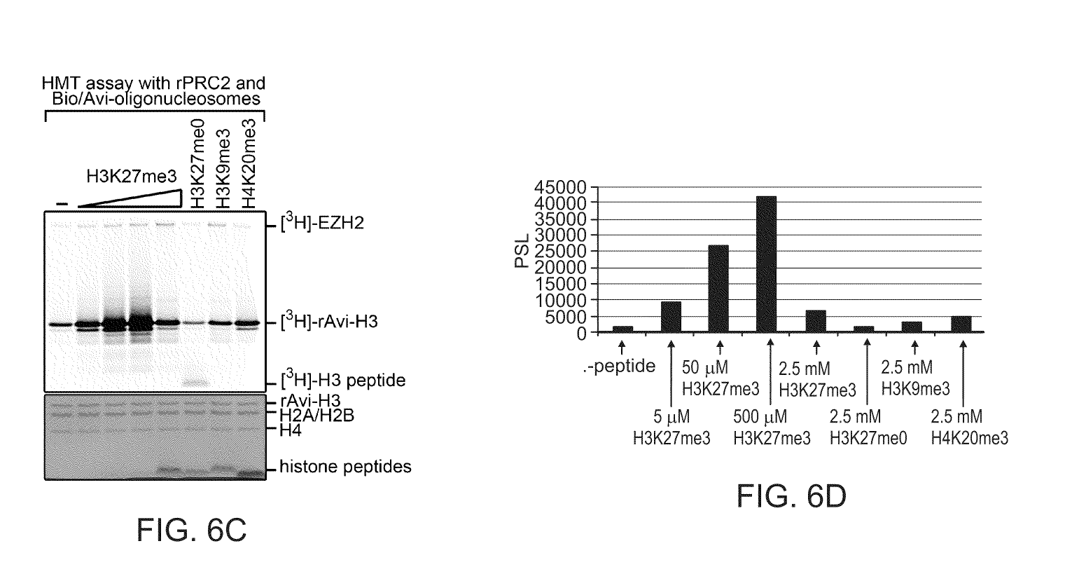

Additional reactions were performed in which methylase activity towards Bio/Avi-H3 or Bio-Avi-oligonucleosomes in the presence of H3K27me3, H3K27me0, H3K9me3, and H4K20me3 was compared. The results, depicted in FIGS. 6A-6D, show that H3K27me3 potently stimulated rPRC2 activity.

### Stimulation by Tri-, Di-, and Mono-Methylated Peptides

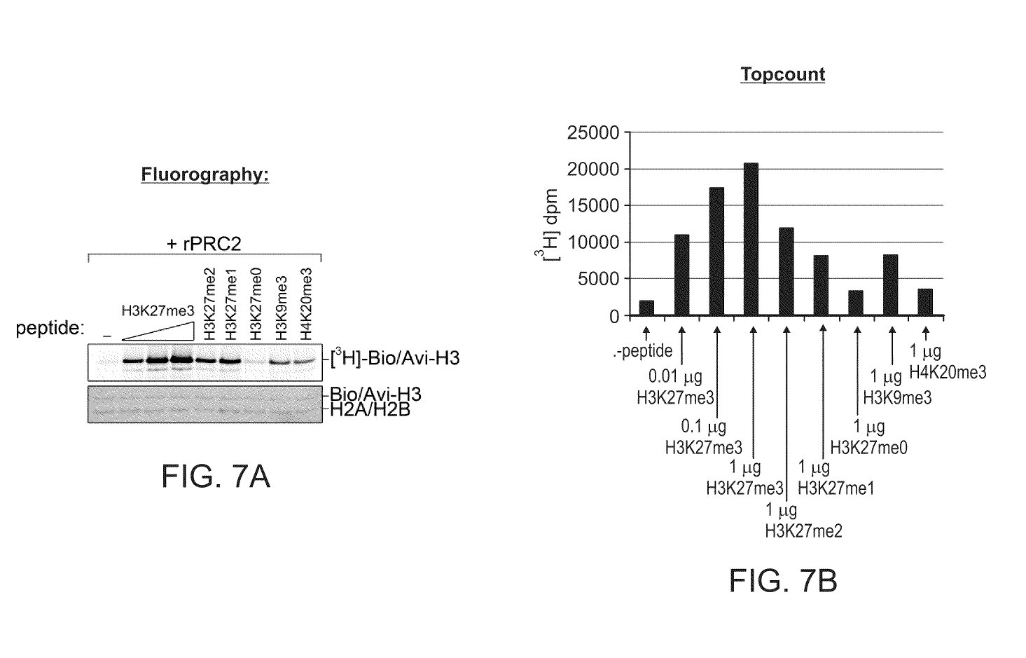

Results of a further experiment are shown in FIGS. 7A and 7B. In this experiment, rPRC2 methylase activity toward Bio/Avi-H3 in the presence of H3K27me3, H3K27me2, H3K26me1, H3K27me0, H3K9me3, and H4K20me3 were compared. The results show that H3K27me3 peptides stimulate rPRC2 methylase activity approximately 11-fold. Dimethylated H3K27 peptide stimulates activity approximately 6-fold. Monomethylated H3K27 stimulates activity approximately 4-fold. Other trimethylated H3 peptides also stimulate activity. Maximal H3K27me3 activity was observed between 0.1-1.0 mg/ml.

### Enzyme Titration and Time Course of Stimulation

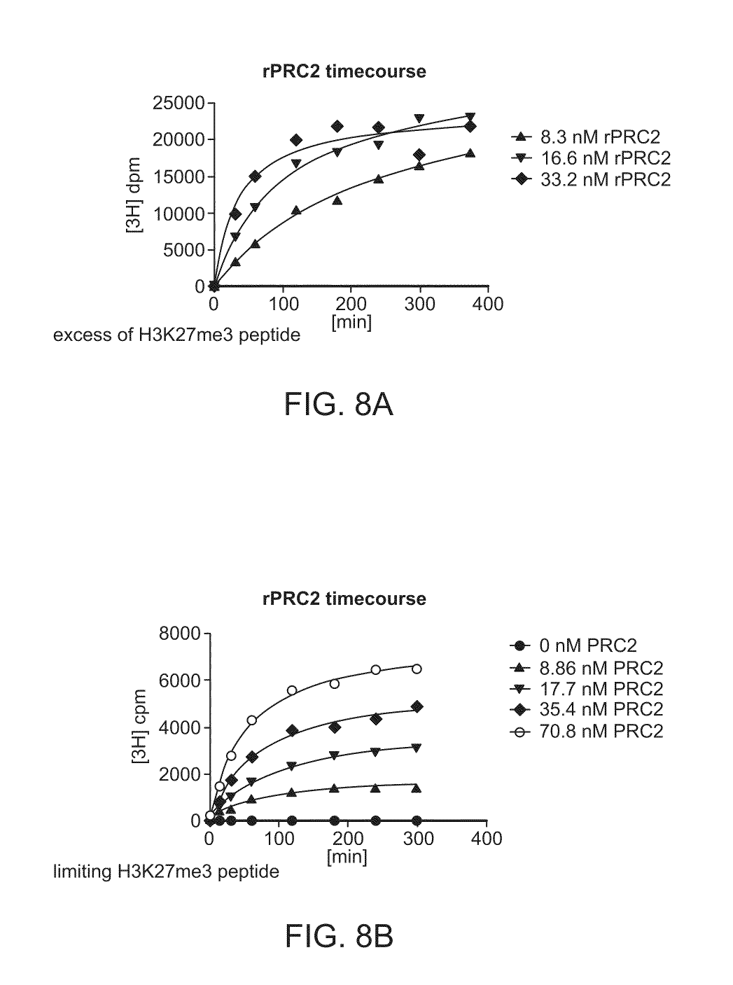

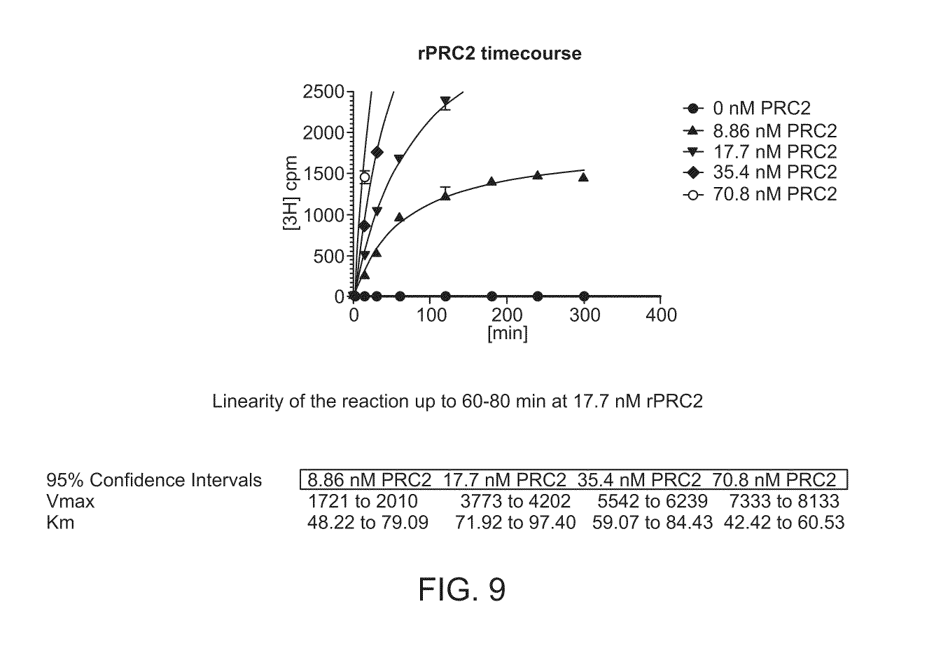

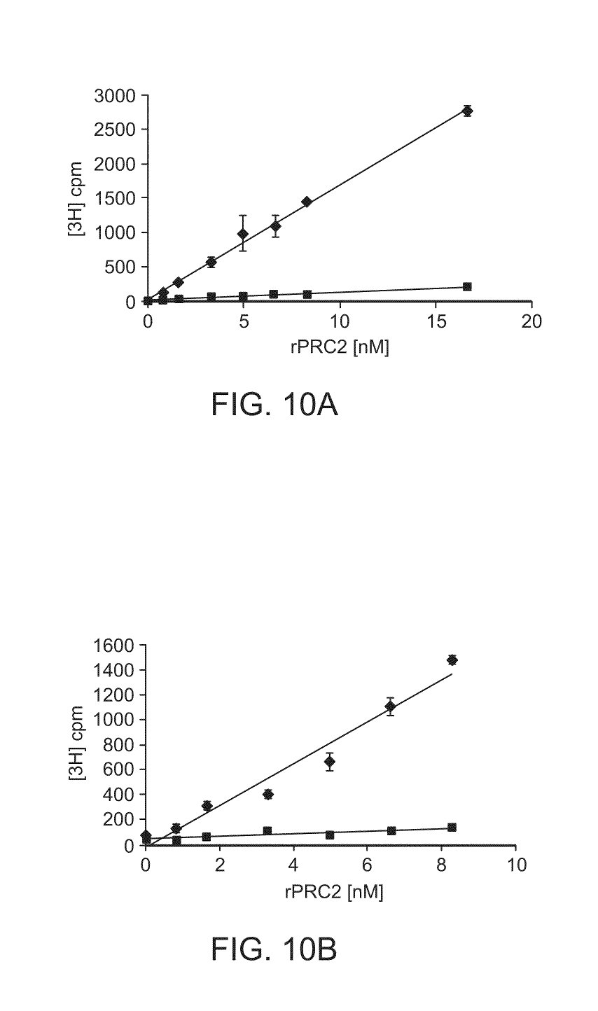

The time course of stimulation of rPRC2 methylation of Bio/Avi-oligonucleosomes by excess and limiting concentrations H3K27me3 peptides was examined. Results are shown in FIGS. 8A and 8B, respectively. Reaction components and conditions are listed in FIG. 8C. Km of rPRC2 was measured and is shown in FIG. 9. Conditions and results of enzyme titration experiments using oligonucleosomes as substrate in the presence of H2K27me3 peptides are shown in FIGS. 10A, 10B and 10C.

### Example 7

### Stimulation of NSD2 Family Members

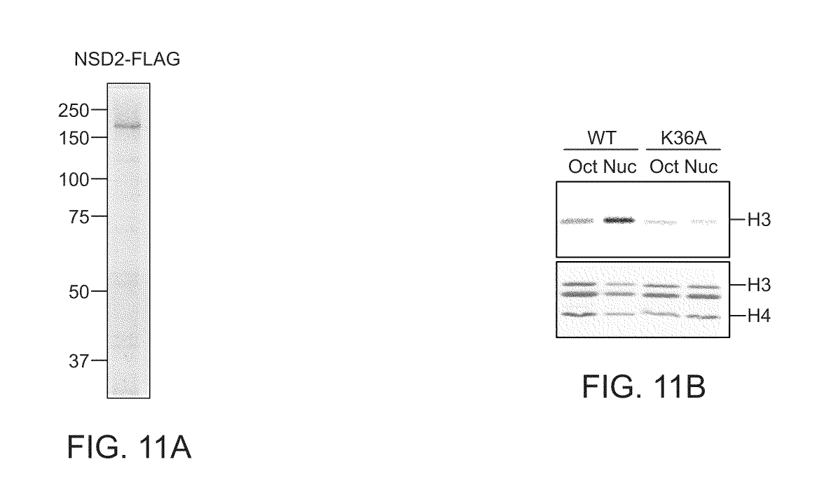

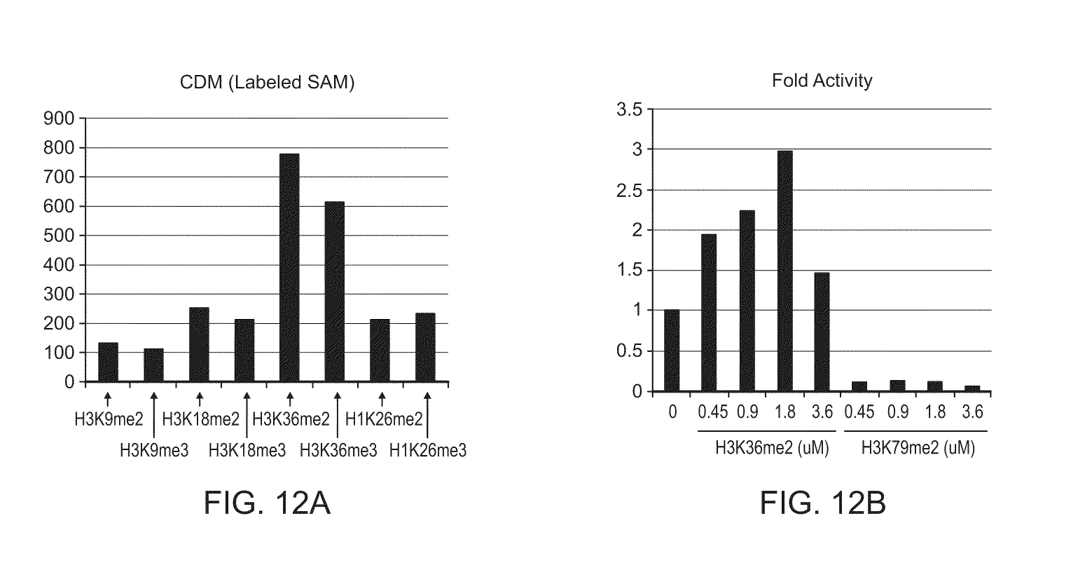

Native NSD2 was purified from 293 cells (FIG. 11A). Methylase activity of NSD2 was evaluated as described in Examples above for EZH2, and it was shown that NSD2 is active towards H3K36 (FIG. 11B). NSD2 methylase activity was next tested in the presence of various histone peptides, including H3K9me2, H3K9me3, H3K18me3, H3K36me2, H3K36me3, H1K26me2, H1K26me3, and H3K79me2. Data are shown in FIGS. 12A and 12B. It was discovered that H3K36me2 and H3K36me3 stimulate NSD2 activity.

### EQUIVALENTS

Those skilled in the art will recognize, or be able to ascertain using no more than routine experimentation, many equivalents to the specific embodiments of the invention described herein. The scope of the present invention is not intended to be limited to the above Description. Alternative methods and materials and additional applications will be apparent to one of skill in the art, and are intended to be included within the following claims:

1. A method of evaluating a test compound, the method comprising:
contacting a histone methyl modifying enzyme and a substrate with a test compound in the presence of a stimulating agent;
evaluating activity of the histone methyl modifying enzyme on the substrate in the presence of the test compound, relative to a control, wherein a change in activity of the histone methyl modifying enzyme in the presence of the test compound, relative to the control, indicates that the test compound is a modulator of the histone methyl modifying enzyme.

2. (canceled)

3. The method of claim 1, wherein the histone methyl modifying enzyme comprises a histone methylase.

4. The method of claim 1, wherein the histone methyl modifying enzyme comprises a histone demethylase.

5. The method of claim 1, wherein the substrate is selected from the group consisting of a peptide, a histone polypeptide, a plurality of histone polypeptides, a nucleosome, an oligonucleosome.

6-9. (canceled)

10. The method of claim 1, wherein the stimulating agent comprises a methylated peptide.

11. The method of claim 10, wherein the methylated peptide is 4-60 amino acids in length.

12. The method of claim 10, wherein the methylated peptide comprises one or more methylated lysine residues.

13. The method of claim 12, wherein the methylated peptide comprises one or more tri-methylated lysine residues.

14. The method of claim 12, wherein the methylated peptide comprises one or more di-methylated lysine residues.

15. The method of claim 12, wherein the methylated peptide comprises one or more mono-methylated lysine residues.

16-17. (canceled)

18. The method of claim 10, wherein the methylated peptide comprises a methylated histone peptide selected from the group consisting of a methylated histone H3 peptide, a methylated histone H4 peptide, and a methylated histone H1 peptide.

19-23. (canceled)

24. The method of claim 18, wherein the methylated histone peptide comprises at least four consecutive amino acids of the following H3 histone peptide sequence:
(SEQ ID NO: 1)
ARTKQTARKSTGGKAPRKQLATKAARKSAPATGESKKPHRYRPGTAAL
REIRRYQKSTEL.

25. The method of claim 24, wherein the H3 histone peptide is methylated on one or more of the following lysine residues: K4, K9, K18, K27, K36, and K79.

26. The method of claim 25, wherein the H3 histone peptide is methylated on K27.

27. The method of claim 25, wherein the H3 histone peptide is methylated on K9.

28. The method of claim 18, wherein the methylated histone peptide comprises at least four consecutive amino acids of the following H4 histone peptide sequence:
(SEQ ID NO: 2)
SGRGKGGKGLGKGGAKRHRKVLRDNIQGITKPAIRRLARRGGVKRISG
LIYEETRGVLKV.

29. The method of claim 28, wherein the H4 histone peptide is methylated on K20.

30. The method of claim 18, wherein the methylated histone peptide comprises at least four consecutive amino acids of the following H1 histone peptide sequence:
(SEQ ID NO: 3)
SETAPAAPAAPAPAEKTPVKKKARKSAGAAKRKASGPPVSELITKAVA
ASKERSGVSLAA.

31. The method of claim 30, wherein the H1 histone peptide is methylated on K25.

32. The method of claim 1, wherein the stimulating agent is present in an amount which stimulates activity of the histone methyl modifying enzyme at least 2-fold.

33-34. (canceled)

35. The method of claim 1, wherein the test compound comprises a small molecule, a peptide, an antibody, or a nucleic acid.

36. The method of claim 1, wherein the methyl modifying enzyme and substrate are contacted with a library of test compounds, and wherein a change in activity of the methyl modifying enzyme in the presence of the library, relative to a control, indicates that the library comprises a modulator of the methyl modifying enzyme.

37-38. (canceled)

39. The method of claim 3, wherein the histone methylase comprises a Polycomb Repressive Complex 2 polypeptide complex.

40. A reaction mixture comprising:
a histone methyl modifying enzyme;
a substrate; and
a stimulating agent, wherein the stimulating agent is present in an amount sufficient to increase activity of the histone methyl modifying enzyme.

41-73. (canceled)

74. The method of claim 5, wherein the plurality of histone polypeptides comprises a histone dimer, a histone tetramer, or a histone octamer.

75. The method of claim 1, wherein the stimulating agent comprises a methylated peptide, and wherein the substrate is selected from the group consisting of a peptide, a histone polypeptide, a plurality of histone polypeptides, a nucleosome, an oligonucleosome.

76. The method of claim 75, wherein the histone peptide is a methylated histone peptide.

77. The method of claim 4, wherein the histone demethylase is selected from the group consisting of GASC1, JARID1C/SMCX and PHF8.
